(19) United States
(12) Patent Application Publication
(10) Pub. No.: US 2011/0262902 A1
(43) Pub. Date: Oct. 27, 2011

(54) CIS-ACTING DIVERSIFICATION ACTIVATOR AND METHOD FOR SELECTIVE DIVERSIFICATION OF NUCLEIC ACIDS

(75) Inventors: Randolph B Caldwell, Munchen (DE); Jean-Marie Buerstedde, Hildesheim (DE); Ulrike Schötz, Viechtach (DE)

(73) Assignee: Heimholz Zentrum Munchen Deutsches Forschungszen- trum fur Gesundheit und Umwelt (GmbH, Neuherberg (DE)

(21) Appl. No.: 12/937,260

(22) PCT Filed: Apr. 9, 2009
(86) PCT No.: PCT/EP09/02680

§ 371 Date: Jun. 2, 2011

(30) Foreign Application Priority Data
Apr. 10, 2008 (EP) 08007120.2

(51) Int. Cl. C12Q 1/68; C12N 5/10; C12N 15/63

(52) U.S. Cl. 435/6.1; 435/440; 435/455; 435/325; 435/349; 435/353; 435/354; 435/320.1

(57) ABSTRACT

The invention relates to identification of a cis-acting diversification activator (DIVAC) that is necessary and sufficient for the activation of diversification in transcription units linked thereto. The invention provides a method for diversification of a target nucleic acid comprising introducing a genetic construct comprising the diversification activator into a recipient cell, wherein the diversification activator is linked to the target nucleic acid.

The present invention relates to a method for selective diversification of a target nucleic acid sequence in a recombinant recipient cell by employing a regulatory sequence from the immunoglobulin locus that activates genetic diversification in linked transcription units.

### INTRODUCTION

Mutated and sequence optimized genes and gene products have extended application in medical, chemical and technical areas. Present strategies for diversification of genes and gene products rely mainly on the generation of a vast number of mutant sequences in vitro, artificial expression and subsequent selection for a desired property. These methods have the drawback that the mutated genes are difficult to express in the physiological context and their mutant repertoire is fixed in time when they are expressed. On the other hand, if genome wide mutagenesis is induced in living cells, this is accompanied by high toxicity due to accidentally lethal mutations and the mutation rates in individual genes are low, i.e., the mutagenesis lacks selectivity.

Accordingly, attempts were made to provide a genetic system that allows for locus specific nucleic acid diversification within living cells. In nature, directed and selective diversification is primarily employed to create diversity within the immunoglobulin genes. Thus, vertebrate B cells are able to diversify their rearranged immunoglobulin (Ig) genes by hypermutation, gene conversion and class switch recombination. All three phenomena require expression of Activation Induced cytidine Deaminase (AID, NC_{—}006088) [Miramatsu et al., 2000; Arakawa et al., 2002; Harris et al., 2002), which most likely initiates Ig gene diversification by deaminating cytidines within the mutating and recombining sequences [Di Noia and Neuberger, 2002; Rada et al., 2004]. A further requirement for hypermutation and switch recombination is the transcription of the Ig genes and the switch regions respectively [Peters and Storb, 1996; Shinkura et al., 2003].

Thus, it was also observed that the inactivation of the E2A transcription factor gene reduces the rate of IgL chain mutations without affecting the levels of surface Ig or AID expression, suggesting that E2A-encoded proteins enhance Ig hypermutation by recruitment of AID to the Ig loci [Schoetz et al., 2006].

Sequence analysis of transcribed non-Ig genes from AID expressing B cells revealed either none or very few mutations compared to Ig genes [Shen et al., 1998]. A recent study of numerous expressed genes in B cells indicated that the number of mutations in wild-type mice is significantly higher than in AID knock-out mice [Liu et al., 2008]. However, the mutation rates for the non-Ig genes in AID expressing B cells were still orders of magnitude lower than for the Ig genes. To explain this difference between Ig and non-Ig genes it was suggested that cis-acting sequences in the Ig loci activate hypermutation possibly by recruiting AID. However, efforts to unambiguously define these sequences for the murine and human Ig loci were unsuccessful [Odegard and Schatz, 2006]. Studies using chimeric reporter genes in transgenic mice indicated that certain Ig enhancers and their surrounding sequences conferred hypermutation activity [Betz et al., 1994; Klotz and Storb, 1996; Klix et al., 1998]. However, deletion of Igk enhancers in knock-out mice did not prevent hypermutation of the Igk gene (CAA36032) [van der Stoep et al., 1998; Inlay, 2006]. At least one murine B cell line [Wang et al., 2004] and AID expressing fibroblasts [Yoshikawa et al., 2002] mutated transcribed transgenes in the absence of nearby Ig locus sequences, further confounding the issue of whether cis-acting regulatory sequences are needed for hypermutation.

Previously the inventors demonstrated that the chicken B cell line DT40 diversifies its rearranged Ig light chain (IgL) gene by gene conversion in the presence of nearby pseudo V (ψV) genes [Arakawa et al., 2002] or by hypermutation, if the ψV genes are deleted [Arakawa et al., 2004; WO 2005/080552]. Both activities strictly depend on the expression of AID. Subsequently, the inventors showed that a Green Fluorescent Protein (GFP, AAB08058) transgene is rapidly diversified by mutations when inserted into the rearranged IgL locus [Arakawa et al., 2008], consistent with the initial finding that the deletion of homologous gene conversion donors leads to hypermutation of the IgL gene [Arakawa et al., 2004]. At the same time, no mutations were found in the highly transcribed Elongation Factor alpha gene (NP_{—}989488) [Arakawa et al., 2004], indicating that hypermutation occurs only within the IgL locus. Also the VpreB3 (NC_{—}006102) or the Carbonic Anhydrase (XP_{—}415218) gene, upstream and downstream neighbors of the IgL locus, respectively showed no sequence heterogeneity in DT40 [Gopal and Fugmann, 2008]. It was observed that a 4.1 kb deletion of the rearranged IgL locus downstream of the enhancer region stopped IgL gene conversion in ψV positive DT40 [Kothapalli et al., 2008], indicating that parts of the IgL locus are necessary for gene conversion. However, the technique used in this study could not be used for a precise identification of small specific diversification active sequences. Moreover, the question whether a putative diversification sequence is sufficient for activation of diversification could also not be resolved.

Thus, none of the known studies either in human, murine or DT40 cells revealed a cis-acting regulatory sequence that is both necessary and sufficient for the Ig locus specificity of AID-dependent genetic diversification. Such a sequence would allow generating diversity of nucleic acid target sequences outside the Ig loci in AID expressing cells.

Therefore, there exists a need for a regulatory sequence that activates diversification in the nucleic acids linked thereto and a method to achieve specific diversification of a target nucleic acid. The present invention satisfies the need and provides further advantages as well.

### SUMMARY OF THE INVENTION

The invention makes available a regulatory nucleic acid molecule, a cis-acting diversification activator (DIVAC), that has the function of activating diversification in transcription units linked thereto. A diversification activator of the invention is derived from the immunoglobulin (Ig) locus of a chicken B cell and comprises a nucleic acid having the sequence SEQ ID NO:1, a fragment of said nucleic acid or a nucleic acid homologous to SEQ ID NO:1 or a fragment thereof, whereby said fragment and said homologue retain the DIVAC function.

Accordingly, the invention provides a method for diversification of a target nucleic acid comprising introducing a genetic construct comprising a diversification activator of the invention into a recipient cell to produce a recombinant recipient cell, wherein said diversification activator is linked to the target nucleic acid.

A target nucleic acid can be supplied to the recipient cell from outside as a transgene, or be part of the recipient cell genome. In each case, it is secured that the target nucleic acid is linked to DIVAC, i.e., DIVAC activates and directs the diversification of the target nucleic acid.

The invention further provides a method for producing a target nucleic acid having a desired activity, comprising the steps of (a) transfecting or infecting a recipient cell one or more times with one or more genetic construct(s) comprising a diversification activator and, optionally, the target nucleic acid to obtain a recombinant recipient cell, (b) identifying a recombinant recipient cell, wherein said diversification activator is linked to said target nucleic acid, (c) propagating and expanding the cell from (b) under conditions appropriate for the expression and diversification of the target nucleic acid, and (d) selecting within the population of cells from (c) individual cells or cell populations containing a mutated target nucleic acid having a desired activity.

The invention further provides a recombinant nucleic acid construct comprising one or more diversification activator(s) of the invention either alone or linked to a target gene.

Further provided is a recombinant cell comprising a diversification activator of the invention integrated into the cell chromosome at a position different from the immunoglobulin (Ig) locus.

### DESCRIPTION OF THE FIGURES

FIG. 1. Hypermutation of the GFP2 reporter at various distances from the rearranged IgL locus. (A) A physical map of the rearranged IgL locus, a targeting construct including the GFP2 reporter and the IgL locus after targeted insertion of the GFP2 reporter. (B) Locations of GFP2 insertion relative to the IgL locus on chromosome 15. The reference point is the insertion site of GFP2 in the IgL^{GFP2} transfectant. (C) FACS analysis of primary transfectants having integrated the transfected construct targeted. (D) Fluctuation analysis of subclones. Each dot represents the analysis a single subclone, and the median of all subclones from the same primary transfectant is indicated by a bar. (E) Targeted integration of GFP2 containing constructs at various distances from the IgL locus into chromosome 15 [22]. (F) Targeted integration of GFP2 containing constructs at the A-MYB, RAD52, BACH2 and Bc16 loci.

FIG. 2. GFP2 hypermutation after deletions and reconstitution of the rearranged IgL locus. (A) Design of deletions and insertions in the rearranged IgL locus using GFP2 targeting constructs. (Upper part) Physical maps of the rearranged IgL locus after the deletion of the ψV locus, a targeting construct including the GFP2 reporter and the locus after targeted integration. (Middle part) Physical maps of the deleted rearranged IgL locus, a targeting construct including the GFP2 reporter and the GFP2 insertion site after targeted integration. (Lower part) Physical maps of the deleted rearranged IgL locus, a targeting construct including the GFP2 reporter together with the ‘W’ fragment and the GFP2 insertion site in the reconstituted IgL locus. (B) FACS analysis of primary transfectants. (C) Fluctuation analysis of subclones. (D) The frequencies of particular nucleotide substitutions within the GFP open reading frame of GFP2.

FIG. 3. Analysis of the ‘W’ fragment deletion series. (A) A physical map of the deleted IgL locus and the aligned targeting constructs leading to the insertion of the GFP2 reporter together with parts of the ‘W’ fragment. (B) FACS analysis of representative primary transfectants. (C) Fluctuation analysis of subclones. Only the letter of the reconstituted fragment and not the full name of the transfectants is indicated.

FIG. 4. Insertions of the GFP2 reporter into non-Ig loci. (A) Chromosomal locations of the GFP2 insertions. (B) FACS analysis of primary transfectants. (C) Fluctuation analysis of subclones.

FIG. 5. Hypermutation of the GFP2 reporter at non-Ig loci in the presence of the ‘W’ fragment. (A) Physical maps of the non-Ig loci, the targeting constructs including the GFP2 reporter together with the ‘W’ fragment and the loci after targeted insertion of the GFP2 reporter. (B) FACS analysis of primary AID positive and AID negative transfectants. (C) Fluctuation analysis of subclones.

FIG. 6. Analysis of the ‘S’ (or, ‘0-4’) fragment 1 kb progressively increasing deletion series. The deletion series consisted of deletions progressively increasing by 1 kb starting from the 5′ as well as from the 3′ end of the sequence. (A) A physical map of the deleted IgL locus and the aligned targeting constructs leading to the insertion of the GFP2 reporter together with parts of the ‘S’ fragment. (B) Fluctuation analysis of subclones.

FIG. 7. Analysis of the ‘0-4’ fragment 200 bp deletion series. The deletion series consisted of consecutive 200 bp deletions starting from the 5′ end of the sequence. (A) A physical map of the ‘0-4’ fragment showing the location of the 200 bp deletions relative to the putative enhancer site. (B) Fluctuation analysis of subclones.

FIG. 8. Analysis of the ‘2-3’ fragment 50 bp deletion series. The deletion series consisted of consecutive 50 bp deletions starting from the 5′ end of the sequence. (A) A physical map of the ‘2-3’ fragment showing the location of the 50 bp deletions relative to the putative enhancer site. (B) Fluctuation analysis of subclones.

FIG. 9. Analysis of the ‘2-3’ fragment 50 bp progressively increasing 5′ deletion series. The deletion series consisted of deletions progressively increasing by 50 bp, starting from the 5′ end of the sequence. (A) A physical map of the ‘2-3’ fragment showing the location of the progressively increasing 50 bp deletions relative to the putative enhancer site. (B) Fluctuation analysis of subclones.

FIG. 10. Analysis of the ‘2-3’ fragment 50 bp progressively increasing 3′ deletion series. The deletion series consisted of deletions progressively increasing by 50 bp starting from the 3′ end of the sequence. (A) A physical map of the ‘2-3’ fragment showing the location of the progressively increasing 50 bp deletions relative to the putative enhancer site. (B) Fluctuation analysis of subclones.

FIG. 11. Reconstitution and multimerisation of the ‘2.2-2.4’ fragment. (A) A physical map illustrating the ‘2.2-2.4’ monomer and multimers used as diversification activator. (B) Fluctuation analysis of subclones.

FIG. 12. Insertion of the ‘2.2-2.4’ fragment at the BACH2 locus. (a) Physical maps of the BACH2 locus, the targeting construct including the GFP2 reporter together with the ‘2.2-2.4’ fragment and the locus after targeted insertion of the GFP2 reporter. The GFP is indicated by an arrow and the ‘2.2-2.4’ fragment is marked by a grey box. (b) Fluctuation analysis of subclones. Only the letter and position of the reconstituted fragment and not the full name of the transfectants is indicated. The median of GFP decrease for each clone is indicated above the bars.

FIG. 13. Analysis of the ‘2.2-2.4’ homologues in turkey and duck. (A) A physical map of the ‘2.2-2.4’ fragment in chicken and its homologous counterparts in turkey and duck. The white area displays the sequence conserved between the species. The black bars indicate the sequence differences between the species. The grey bars are conserved sequences in duck and turkey but differ from chicken. (B) Fluctuation analysis of the subclones (n=24) of one primary clone. The median of decreased green fluorescence for each clone is written above the bars.

FIG. 14. Position of binding motifs relative to the 9.8 kb (‘W’) fragment. Binding motifs for E2A transcription factors, NF-κB factors and ISRE are shown. The motifs are annotated with their name and their exact nucleotide position. The position of important deletion fragments within ‘W’ is described: ‘0-4’ (green box), ‘2-3’ (red box), ‘2.2-2.4’ (orange box), ‘3-4’ (light blue box), and ‘N’ (dark blue box).

### DETAILED DESCRIPTION OF THE INVENTION

The present invention makes available an improved method for genetic diversification of a target nucleic acid within a living cell. The method is based on the discovery of a regulatory nucleic acid molecule, a cis-acting diversification activator (DIVAC), that is essential and sufficient for the activation of genetic diversification of a nucleic acid that is linked thereto.

For the purpose of the invention the following definitions apply.

“Genetic diversification” is alteration of individual nucleotides or stretches of nucleotides in a target nucleic acid. Genetic diversification according to the invention preferably occurs by hypermutation. In the event that in the method of the invention homologous donors for the target nucleic acid are provided, genetic diversification may occur by gene conversion or a combination of hypermutation and gene conversion.

“Hypermutation” diversifies a target nucleic acid primarily by single nucleotide substitutions. Preferably, hypermutation refers to a rate of mutation of between 10⁻⁵ and 10⁻³ bp⁻¹ generation⁻¹ which at least 100 fold higher than the background mutation rate in non-hypermutating genes within the same cell.

“Gene conversion” is a phenomenon in which sequence information is transferred in unidirectional manner from a homologous gene conversion donor to a gene conversion target sequence. Gene conversion may be the result of a DNA polymerase switching templates and copying from a homologous sequence, or the result of mismatch repair (nucleotides being removed from one strand and replaced by repair synthesis using the other strand) after the formation of a heteroduplex. An example of natural gene conversion is the diversification of the rearranged VJ IgL segments by nearby pseudo-V gene conversion donors in certain species.

In many vertebrates, hypermutation and gene conversion generate diversity within the immunoglobulin V(D)J segment of B cells. Hypermutation takes place in the germinal centers of such species as mouse and human following antigen stimulation. Gene conversion takes place in primary lymphoid organs like the Bursa of Fabricius or the gut-associated lymphoid tissue in such species as chicken, cow, rabbit, sheep and pig independent of antigen stimulation.

“Diversification activator” is a cis-acting regulatory sequence that activates the genetic diversification of neighboring transcription units which behave as target nucleic acids.

“Target nucleic acid” is a nucleic acid molecule subjected to genetic diversification. The target nucleic acid is preferably a transgene and may comprise one or more transcription units encoding gene products. The target nucleic acid may also be a non-coding transcription unit or an endogenous gene whose diversification is activated by the nearby insertion of a diversification activator. The target nucleic acid altered as a result of DIVAC-related diversification is also called mutant nucleic acid.

“Transgene” is an exogenous nucleic acid molecule that is inserted into the recipient cell, such as by transfection, transduction, or infection. For example, a transgene may comprise a heterologous transcription unit which may be inserted into the genome randomly or at a defined location by targeted integration. For the purpose of the invention the transgene is preferably located next to a diversification activator thereby becoming a target nucleic acid.

“Targeted integration” is integration of a transfected nucleic acid construct comprising a nucleic acid sequence homologous to an endogenous nucleic acid sequence by homologous recombination into the endogenous locus. Targeted integration allows to directly insert any nucleic acid into a defined chromosomal position.

“Recombinant recipient cell” refers to a cell that is engineered for genetic diversification of a target nucleic acid. Preferably, the recipient cell expresses AID and is the recipient of the transgene linked to the diversification activator.

“Selection” refers to the determination of the presence of sequence alterations in the target nucleic acid that result in a desired activity of the gene product encoded by the target nucleic acid or in a desired activity of the regulatory function of the target nucleic acid.

Method for Diversification of a Target Nucleic Acid

In one aspect, there is provided a method for diversification of a target nucleic acid comprising introducing a genetic construct comprising a diversification activator into a recipient cell to produce a recombinant recipient cell,
wherein said diversification activator is linked to the target nucleic acid,
wherein said diversification activator is selected from the group consisting of a nucleic acid having the sequence SEQ ID NO:1, a fragment of said nucleic acid and a nucleic acid homologous to said nucleic acid or said fragment, and
wherein said diversification activator has the function of activating genetic diversification in a nucleic acid linked thereto.

In one embodiment, the diversification takes place by the process of somatic hypermutation. A preferred rate of somatic hypermutation is between 10⁻⁵ and 10⁻³ bp⁻¹ generation⁻¹.

In another embodiment, the target nucleic acid is in addition linked to at least one nucleic acid capable of serving as a gene conversion donor for the target nucleic acid, and the diversification takes place by a process involving both somatic hypermutation and gene conversion.

Diversification Activator

A diversification activator (DIVAC) of the invention is derived from the immunoglobulin (Ig) locus of a chicken B cell. In one embodiment, DIVAC is a 9.8 kb fragment (also called “W” fragment) of the rearranged IgL locus extending from the IgL transcription start site to the 3′ end of the carbonic anhydrase gene. The 9.8 kb fragment maps to Gallus gallus chromosome some chr15:8165070-8176699.

The 9.8 kb fragment isolated from the chicken B cell line DT40 has the nucleotide sequence SEQ ID NO:1. In another embodiment, DIVAC of the invention is a fragment of the 9.8 kb fragment that retains the DIVAC function of activating diversification of a nucleic acid linked thereto. The DIVAC fragment may be as short as 50 nucleotides and as long as 5000 nucleotides. For example, the length of DIVAC may be at least 50 nucleotides, at least 100 nucleotides, at least 200 nucleotides, at least 300 nucleotides, at least 400 nucleotides, at least 500 nucleotides, at least 600 nucleotides, at least 700 nucleotides, al least 800 nucleotides, at least 900 nucleotides, at least 1000 nucleotides, at least 1250 nucleotides, at least 1500 nucleotides, at least 1750 nucleotides, at least 2000 nucleotides, at least 2250 nucleotides, at least 2500 nucleotides, at least 2750 nucleotides, at least 3000 nucleotides, at least 3250 nucleotides, at least 3500 nucleotides, at least 3750 nucleotides, at least 4000 nucleotides, at least 4250 nucleotides, at least 4500 nucleotides, at least 4750 nucleotides, at least 5000 nucleotides at least 5250 nucleotides, at least 5500 nucleotides, at least 5750 nucleotides, at least 6000 nucleotides, at least 6250 nucleotides, at least 6500 nucleotides, or at least 6750 nucleotides. The length of DIVAC may be 100, 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, 5250, 5500, 6000, 6250, 6500, 6750, or 7000 nucleotides.

Specific DIVAC fragments may be defined on the basis of the oligonucleotides listed in Table 2 that may be used for PCR amplification of individual fragments. DIVAC fragments may also be defined on the basis of the nucleotide positions within the 9.8 kb fragment (SEQ ID NO:1).

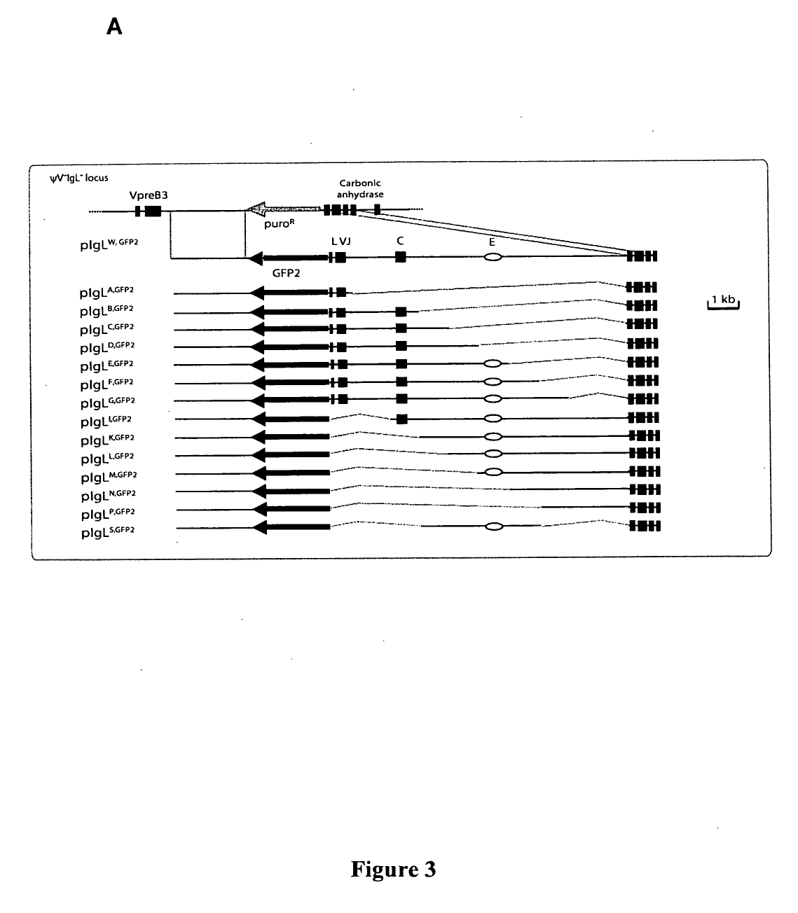

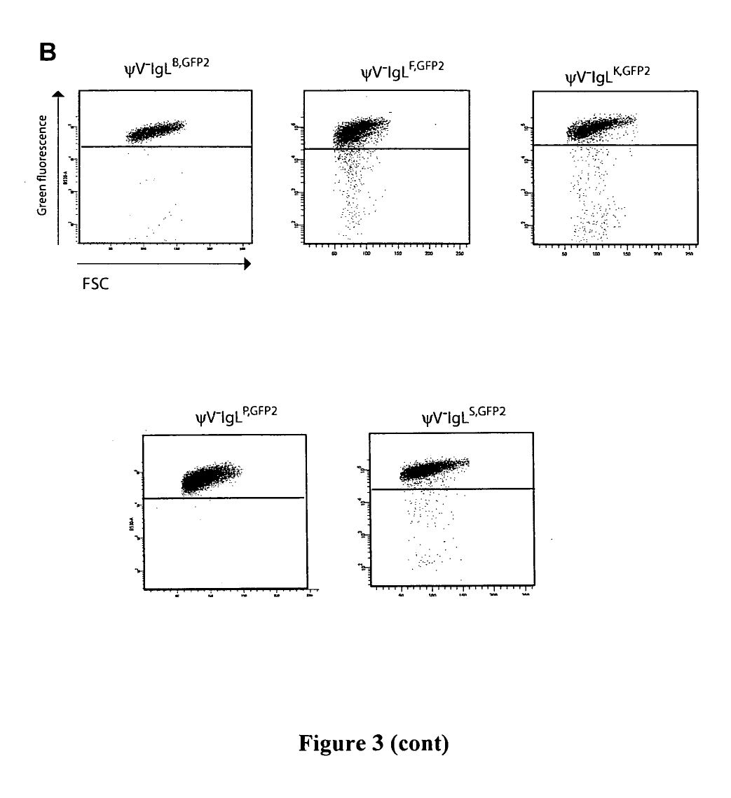

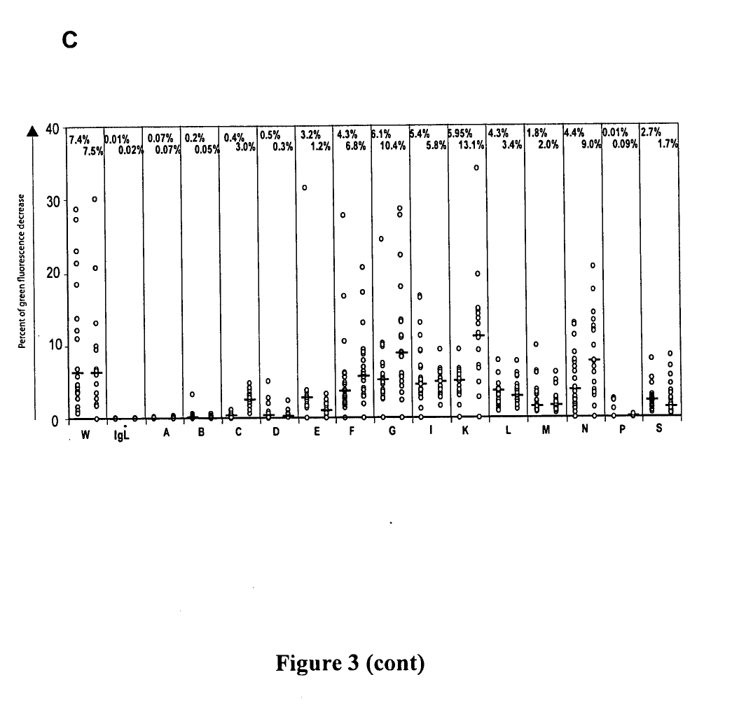

Thus, DIVAC may have the nucleotide sequence between positions 1-7099, 1-7799, 2184-9784, 3098-9784, 4102-9784, 5114-9784, 6107-9784, or 3098-7099 of SEQ ID NO:1. These sequences correspond to the fragments designated F, G, I, K, L, M, N and S, respectively, on FIG. 3A. DIVAC may be a fragment of a length between 5 kb and 1 kb having the nucleotide sequence between positions 1000-3000, 2825-6082, 2000-3000, 2000-4000, 4000-9784, 4000-7000, 4000-6000, 5000-9784, 5000-8000, 5000-7000, 6000-9784, 6000-8000, 8000-9784, 8500-9784 of SEQ ID NO:1. DIVAC may be a shorter fragment of a length between 1 kb and 200 bp having nucleotide sequence between positions 2825-3625, 5000-6000, 6000-7000, 9000-9784, 5114-6106 (‘2-3’), 6107-7099 (‘3-4’), 9509-9802 (‘9.6-9.8’), or 5288-5485 (‘2.2-2.4’). It is preferred that DIVAC comprises or consists of a fragment between positions 6107-9784 (‘N’), 3098-7099 (‘S’), 5288-5485 (‘2.2-2.4’), or 9509-9802 (‘9.6-9.8’) of SEQ ID NO:1. It is also preferred that DIVAC contains an Ig enhancer mapping at position between about 5000 and 6000 on the 9.8 kb (SEQ ID NO:1) scale.

DIVAC can also be a functional homologue of SEQ ID NO:1 or a fragment thereof from the chicken Ig heavy chain (IgH) locus or from the immunoglobulin locus of other avian or mammalian species. It is preferred that the DIVAC homologue is derived from an organism that has the immunoglobulin locus organized similar to the chicken Ig locus. Thus, DIVAC may be a sequence corresponding to SEQ ID NO:1 or a fragment thereof from various birds. For example, DIVAC may be derived from the turkey IgL and have the sequence of SEQ ID NO:117 (homologue of the 9.8 kb fragment), or SEQ ID NO:115 (homologue of the ‘2.2-2.4’ fragment). Alternatively, DIVAC may be derived from the duck IgL and have the sequence SEQ ID NO:118 (homologue of the 9.8 kb fragment), or SEQ ID NO:116 or 119 (homologue of the ‘2.2-2.4’ fragment). It is preferred that DIVAC has the sequence between positions 6107-9784 (‘N’), 3098-7099 (‘S’), 5288-5485 (‘2.2-2.4’), or 9509-9802 (‘9.6-9.8’), corresponding to SEQ ID NO:1 in other organisms.

DIVAC may also be derived from the Ig locus of such organisms as pig, sheep, cow or rabbit, known to diversify their immunoglobulin genes by gene conversion. However, DIVAC may also be a functional homologue of SEQ ID NO:1 or fragment thereof from other mammals such as mouse or human that diversify their immunoglobulin genes by hypermutation.

According to the invention, the DIVAC function of activating diversification of a linked target nucleic acid is defined by an at least 5-fold, 10-fold, 20-fold, 50-fold or 100-fold increase of the diversification rate of the target nucleic acid in the presence of DIVAC compared to that in the absence of DIVAC.

DIVAC functional homologues may further be characterized by their structural homology to the 9.8 kb fragment (SEQ ID NO:1) or fragments thereof as defined above, whereby the homologue retains the DIVAC function of activating diversification of a nucleic acid linked thereto. For example, DIVAC of the invention shares at least 50%, at least 60%, at least 70%, at least 80%, at least 90%, or at least 95% sequence identity with SEQ ID NO:1 of a fragment thereof. For example, the sequence homology between the duck and chicken ‘2.2-2.4’ fragments is about 85%.

To increase the rate of diversification, DIVACs of the invention may be combined. For this purpose, more than one copy of DIVAC may be provided to a recipient cell in form of tandem repeats. It is preferred that such sequences exhibit an additive effect. For example, a repeat may comprise two or more copies of the ‘2.2-2.4’ fragment (SEQ ID NOs: 114, 115, 116 or 119). Alternatively, different DIVAC sequences may be combined. It is preferred that such sequences exhibit a cumulative effect. For example, ‘S’ and ‘N’, ‘2.2-2.4’ and ‘3-4’, ‘3-4’ and a fragment between positions 8000-9000, ‘2.2-2.4’ and a fragment between positions 8000-9000, ‘2.2-2.4’ and ‘9.6-9.8’; ‘3-4’ and a fragment between positions 8000-9000 and ‘9.6-9.8’, a fragment between positions 2825-3625 and ‘0-4’, or a fragment between positions 2825-3626 and ‘0-4’ may be combined to increase the diversification activity.

Target Nucleic Acid

A target nucleic acid of the invention is an arbitrary nucleic acid whose diversification is desired. In the method of the invention, the target nucleic acid is preferably transcribed. In one embodiment, the target nucleic acid encodes a protein. For example, a target nucleic acid encodes an immunoglobulin chain, a selection marker, a DNA-binding protein, an enzyme, a receptor protein, or parts thereof. It is preferred that the target nucleic acid is a human immunoglobulin gene. The method of the invention would thus allow to produce an immunoglobulin gene encoding an immunoglobulin with a specific binding activity such as high specificity and/or high affinity to an antigen. It is also preferred that the target nucleic acid is a fluorescent protein such as Green Fluorescent Protein (GFP) or Yellow Fluorescent Protein (YFP). In this embodiment, the method of the invention would allow to produce a fluorescent protein with a new and different absorption/emission wave length.

In another embodiment, the target nucleic acid possesses a regulatory activity. For example, the target nucleic acid may be a transcription regulatory element such as promoter or enhancer, or encode an interfering RNA molecule for use in specific gene suppression by RNA interference. In this embodiment, a reporter, which is influenced by the target nucleic acid, is required for the identification of a recombinant recipient cell bearing the target nucleic acid of interest.

In one embodiment, the target nucleic acid is an exogenous nucleic acid. An exogenous nucleic acid may be provided to the recipient cell together with DIVAC on the same genetic construct, or, alternatively, on a different genetic construct or constructs. In this embodiment, the genetic construct(s) preferably comprise(s) a selectable marker gene that allows for selection of cells comprising the transgene and/or DIVAC. The selectable marker gene may subsequently be removed by recombination, for example using a cre-recombinase system, or inactivated by other means. It is preferred that the target nucleic acid and DIVAC stably integrate into the cell chromosome. Alternatively, the target nucleic acid and DIVAC may remain in the recipient cell extrachromosomally, for example, on an autonomously replicating viral vector such as an EBV-derived vector.

In another embodiment, the target nucleic acid is a priori a part of the cell chromosome, i.e., an endogenous nucleic acid. It is preferred that the endogenous nucleic acid is not an immunoglobulin gene or a part thereof. In this embodiment, only DIVAC is provided to the cell and it is secured that DIVAC integrates into the cell chromosome in the vicinity of the target nucleic acid.

According to the invention, the target nucleic acid is linked to DIVAC. This implies that the two nucleic acids are on the same chromosome in a position relative to each other that ensures that DIVAC activates and directs the diversification of the target nucleic acid. Generally, it is understood that the diversification activity of DIVAC diminishes with the increase in the distance to the nucleic acid to be diversified. It is therefore preferred that the distance between DIVAC and the target nucleic acid is no more than 150 kb, preferably no more than 125 kb, preferably less than 120 kb, less than 100 kb, less than 70 kb, less than 50 kb, less than 30 kb, less than 10 kb, less than 5 kb, or less than 3 kb.

According to the invention, the target nucleic acid and/or DIVAC may be integrated into the chromosome of a recipient cell at a defined or random location. In one embodiment, a genetic construct comprising an exogenous target nucleic acid and DIVAC is integrated into the cell chromosome at a random location.

In another embodiment, a first genetic construct comprising an exogenous target nucleic acid, i.e., a transgene, is integrated at a defined chromosomal position and a second genetic construct comprising DIVAC is integrated at a chromosomal position that ensures that DIVAC activates and directs diversification of the transgene. It is preferred that DIVAC is integrated in the vicinity of the transgene, preferably not farther than 150 kb or 125 kb or 100 kb away therefrom, preferably less than 120 kb, less than 100 kb, less than 70 kb, less than 50 kb, less than 30 kb, less than 10 kb, less than 5 kb, or less than 3 kb.

In a further embodiment, the target nucleic acid is endogenous, and a construct comprising DIVAC is integrated at a chromosomal position that ensures that DIVAC activates and directs diversification of the target nucleic acid. It is preferred that DIVAC is integrated in the vicinity of the target nucleic acid, preferably not farther than 150 kb or 125 kb or 100 kb away therefrom, preferably less than 120 kb, less than 100 kb, less than 70 kb, less than 50 kb, less than 30 kb, less than 10 kb, less than 5 kb, or less than 3 kb.

Integration at a defined chromosomal position (targeted integration) is achieved by including into the respective genetic construct nucleic acid fragments whose sequence is identical or homologous to the nucleic acid sequence at the integration site. The ability of a cell to integrate genetic constructs by targeted integration is an intrinsic property of the cell. Cells of different species and origins possess this property to a varying degree. For example, targeted integration is an infrequent event in a mouse cell. In contrast, the ALV-transduced chicken B cell line DT40 integrates genetic constructs preferably by targeted integration at a defined chromosomal position.

Recipient Cell

A recipient cell of the invention is a eukaryotic cell expressing activation-induced deaminase (AID) or a functional equivalent thereof. It is preferred that the recipient cell is a B lymphocyte from a vertebrate species, in particular an avian or mammalian B cell. For example, it is a B cell from chicken, rabbit, sheep, cow, pig, mouse or rat. It is further preferred that the recipient cell is a chicken B lymphocyte or derived thereof, in particular the ALV-transduced chicken B cell line DT40 or a derivative thereof.

A recombinant recipient cell of the invention is a recipient cell comprising a diversification activator of the invention as a transgenic (i.e., exogenous) nucleic acid, and a target nucleic acid.

In one embodiment, the recombinant recipient cell expresses the target nucleic acid in a manner that facilitates selection of cells comprising mutants of said target nucleic acids having a desired activity. The selection may be a direct selection for the activity encoded or otherwise determined by the target nucleic acid within the cell, on the cell surface or outside the cell. Alternatively, the selection is an indirect selection for the activity encoded by a reporter nucleic acid or otherwise determined by a reporter.

Trans-Acting Regulatory Factors

According to the invention, diversification of the target nucleic acid may be modulated by, a trans-acting regulatory factor. For example, the trans-acting regulatory factor may be a DNA repair or recombination factor, a DNA polymerase, a transcription factor, an uracil DNA glycosylase, a factor involved in chromosomal organization. The diversification process may also be initiated and terminated by a trans-acting regulatory factor such as activation-induced deaminase (AID). For example, AID may be conditionally expressed and its expression may be switched off once the targeted nucleic acid with a desired property is obtained. This would prevent further mutations that may result in a loss of the optimal property. Similarly, other regulatory factors may be added to or expressed in the recipient cell.

trans-acting factors may bind to respective motifs within a cis-regulatory diversification activation sequence such as DIVAC or by binding to other proteins within the diversification complex formed around the target gene. For example, DIVAC of the invention comprises E-Box, NF-kB and ISRE motifs. Alternatively, trans-regulatory factors may be targeted to the gene or locus of interest by tethering molecules fused thereto that would bind to their respective binding motif integrated within in the vicinity of the gene or within the locus.

Method of Preparing Nucleic Acids and Proteins with Desired Activity

In another aspect, the invention provides a method for preparing a target nucleic acid having a desired activity, comprising the steps of:
(a) introducing into a recipient cell one or more nucleic acid construct(s) comprising a diversification activator of the invention and, optionally, the target nucleic acid to obtain a recombinant recipient cell,
(b) identifying a recombinant recipient cell, wherein said diversification activator is linked to said target nucleic acid,
(c) propagating and expanding the cell from (b) under conditions appropriate for the expression and diversification of the target nucleic acid, and
(d) selecting within the population of cells from (c) individual cells or cell populations containing a mutated target nucleic having a desired activity.

In one embodiment, steps (c) and (d) of the method are iteratively repeated.

In another embodiment, the method may include an additional step (e) comprising determining the sequence of the mutated target nucleic acid from the cells selected in (d).

In a further embodiment, the method additionally includes a step (f) comprising terminating the diversification. The diversification may, for example, be switched off by down-regulation of the expression of a trans-acting regulatory factor such as activation-induced deaminase (AID) or by the removal of the diversification activator. For this purpose, switchable promoters such as tet may be used.

In step (a), the nucleic acid constructs of the invention may be transfected into the recipient cell. Alternatively, the recipient cell may be transduced or infected with the construct.

In step (d), a variety of selection procedures may be applied for the isolation of mutants having a desired property. For example, cells expressing immunoglobulin molecules with improved or novel binding specificity may be selected by Fluorescence Activated Cell Sorting (FACS), cell separation using magnetic particles, antigen chromatography methods or other known cell separation techniques.

In one embodiment, the target nucleic acid and DIVAC(s) are on the same nucleic acid construct. Alternatively, the target nucleic acid and DIVAC(s) are on different constructs. In this embodiment, the genetic locus containing the target nucleic acid to be diversified may be constructed by more than one round of transfection. In a further embodiment, the target nucleic acid is part of the cell chromosome and the construct or constructs comprise(s) one or more DIVACs.

Recombinant Nucleic Acid Construct

In a third aspect, the invention provides a recombinant nucleic acid construct comprising a diversification activator of the invention. The construct may be a plasmid, a virus, a virus-derived vector such as an integrating or autonomously replicating vector. The construct may comprise one or more copies of DIVAC or a combination of different DIVACs.

Recombinant Cell

In a fourth aspect, the invention provides a recombinant cell comprising a diversification activator of the invention as a transgenic (exogenous) sequence. In one embodiment, the cell is a B lymphocyte. A B cell maybe from a vertebrate species such as bird, pig, cow, sheep, rabbit, mouse, or human. It is preferred that a B cell is from chicken. It is highly preferred that the recombinant cell of the invention is a cell of the DT40 cell line.

Excluded from the scope of the invention are highly unlikely embodiment, wherein the diversification activator is integrated into the chromosome of a cell and replaced part of the chromosome identical to the diversification activator, such that the resulting cell is not distinguishable from a natural cell.

The invention is illustrated by the following examples.

### EXAMPLES

### Example 1

### A Hypermutation Reporter Based on GFP Expression

Inventors previously demonstrated that a GFP transgene in DT40 rapidly accumulated mutations, if integrated at the position of the promoter of the rearranged IgL [Arakawa et al., 2008]. The hypermutation activity depended on AID expression and could be visualized by the appearance of cells displaying decreased green fluorescence due to detrimental GFP mutations.

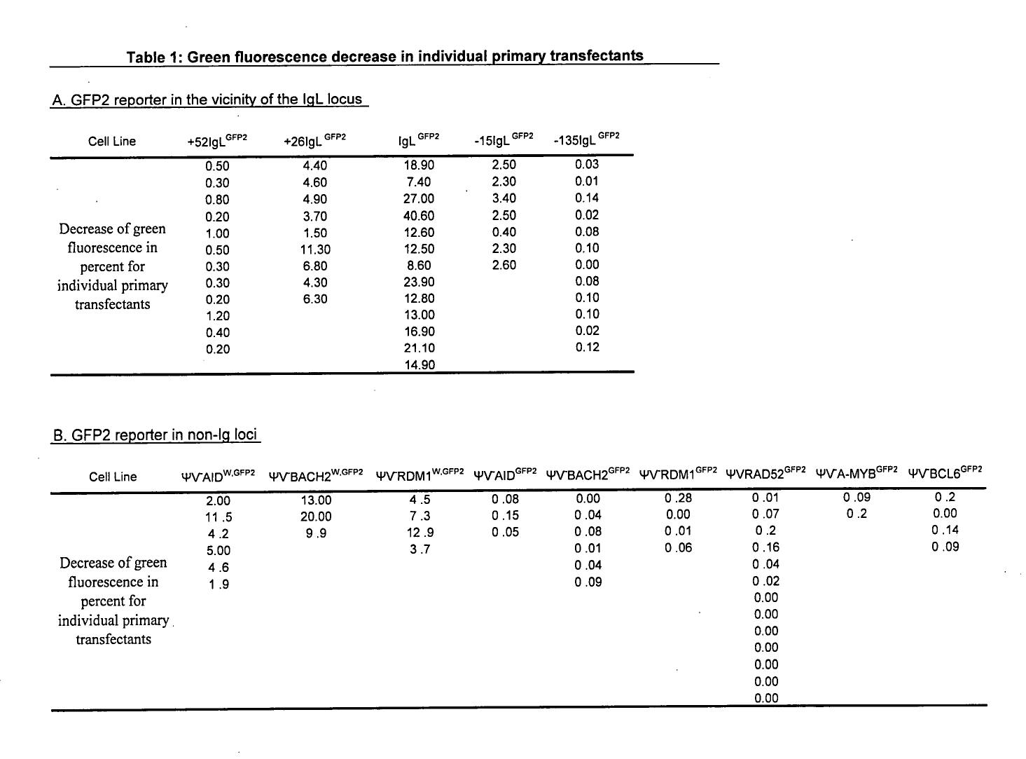

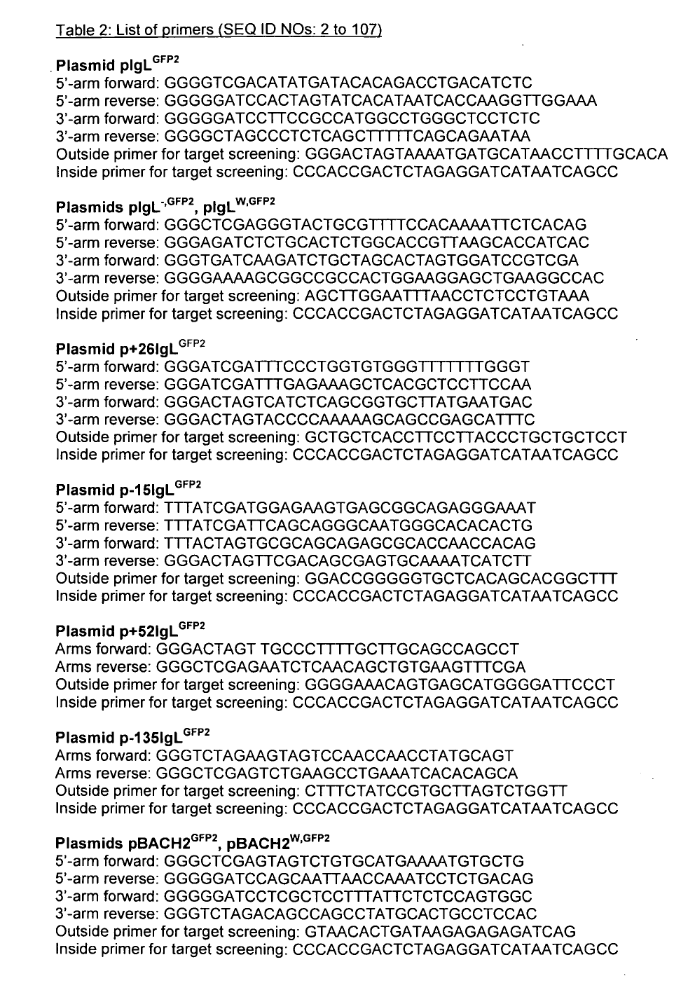

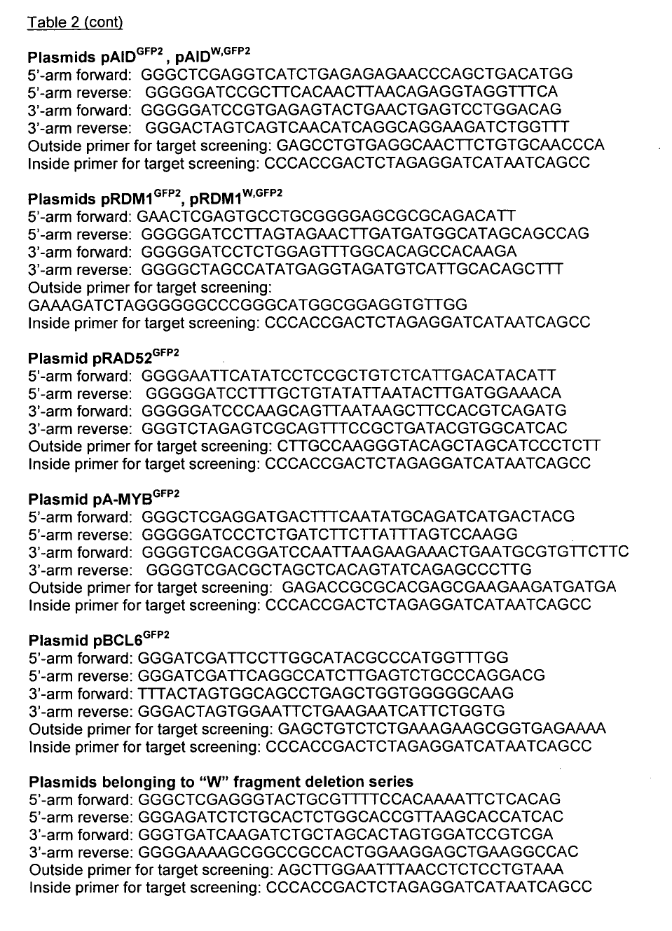

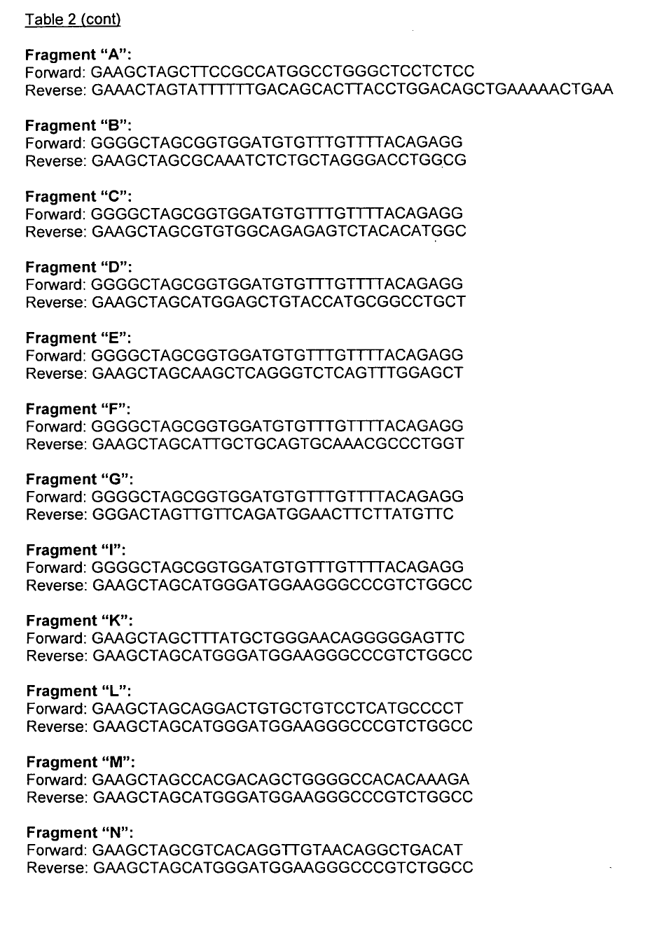

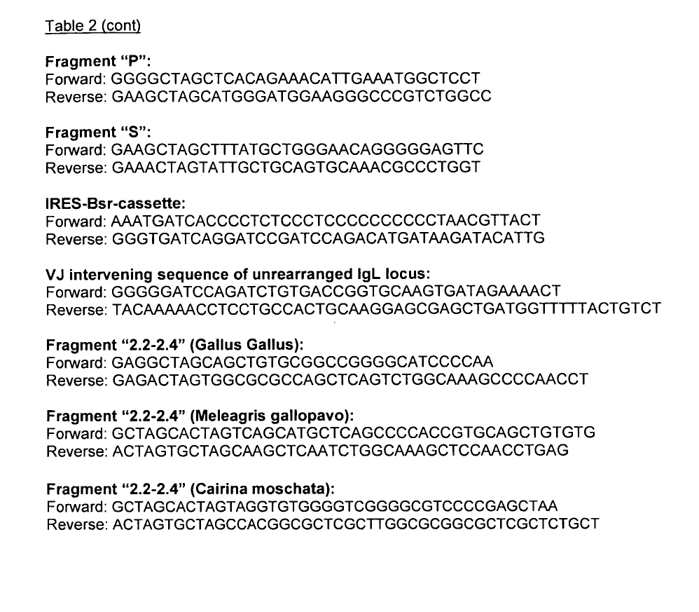

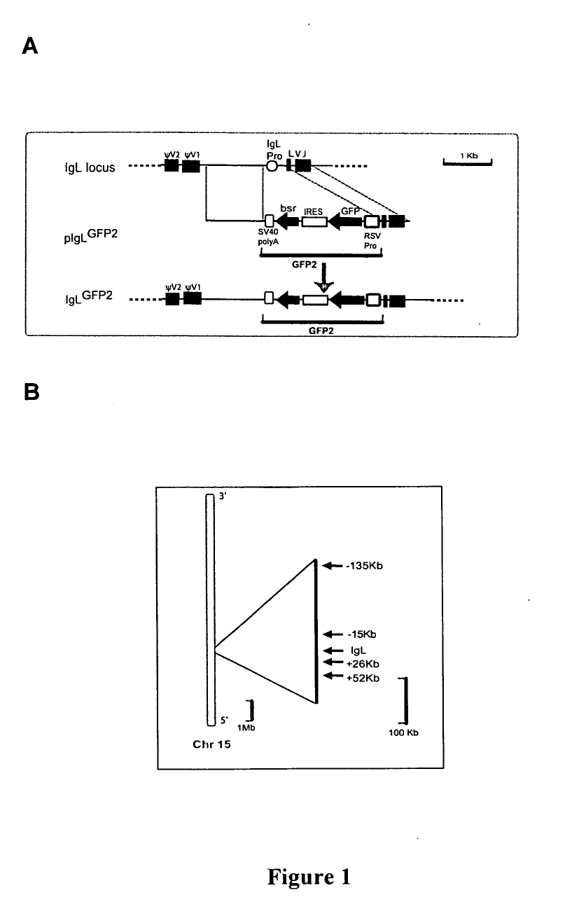

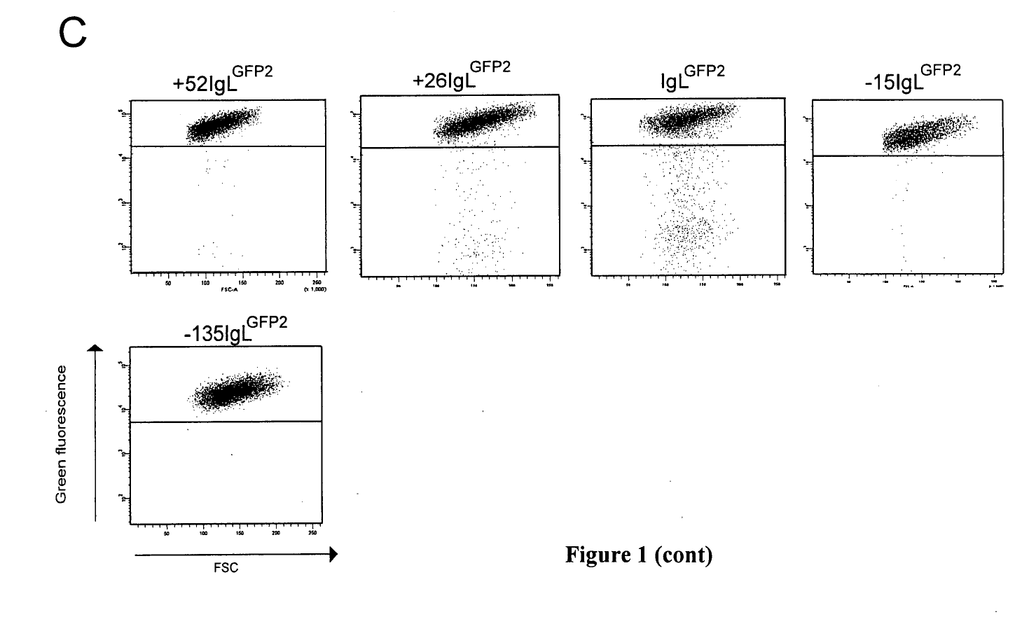

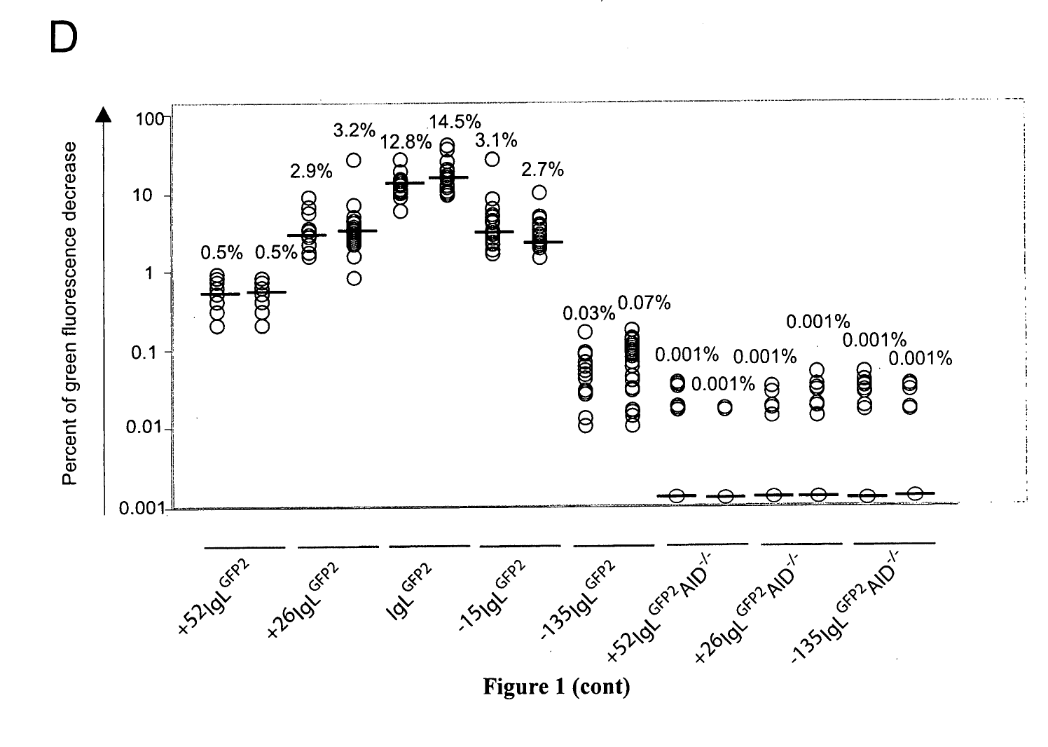

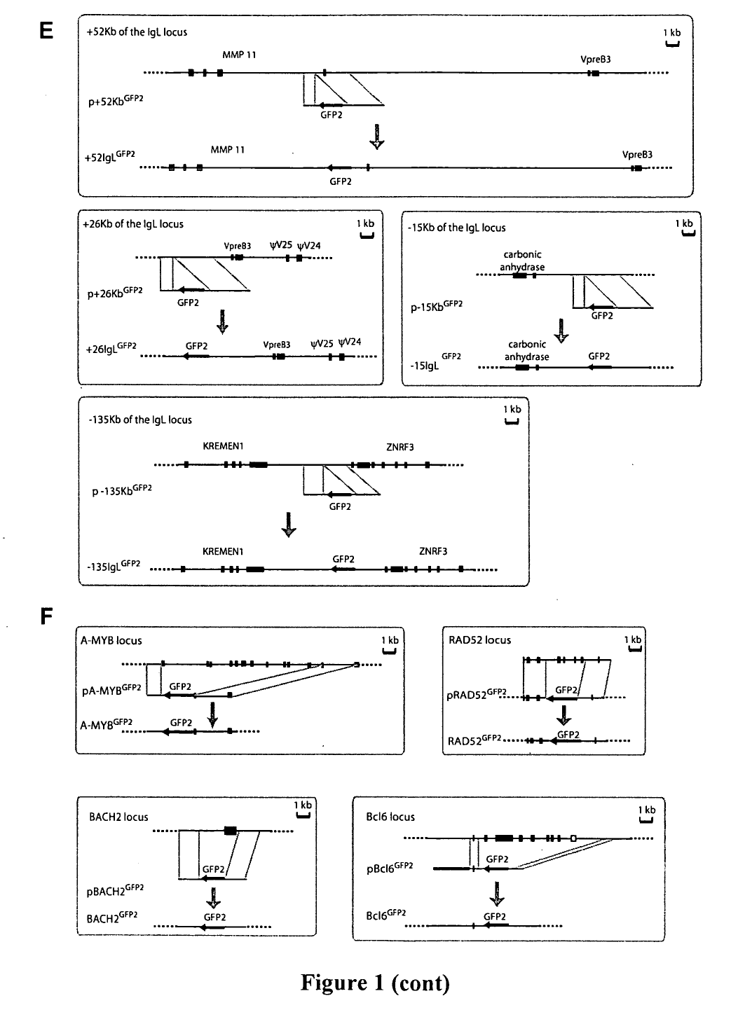

To exploit this phenomenon a new expression cassette named GFP2 was designed which consisted of the strong RSV promoter followed by the GFP coding region, an internal ribosome entry site (IRES), the blasticidin resistance gene (P19997) and the SV40 polyadenylation signal. GFP2 was incorporated into the targeting construct pIgL^{GFP2} (FIG. 1A) in the opposite transcriptional orientation of the IgL gene to minimize interference between transcription and post-transcriptional regulation of the GFP2 transgene and the IgL gene.

Transfection of pIgL^{GFP2} into the conditionally AID expressing clone AID^{R2} yielded a number of transfectants named IgL^{GFP2} in which targeted integration had substituted the IgL promoter by the GFP2 transgene. Fluorescence activated cell sorting (FACS) analysis of subclones from two independent primary transfectants revealed median values of 12.8% and 14.5% decreased green fluorescence (FIGS. 1C and 1D). The result confirms the results of inventors' previous study indicating that the GFP2 transgene is mutated at high rate within the rearranged IgL locus and that cell populations with decreased green fluorescence can be used to quantify this hypermutation activity.

### Example 2

### Hypermutation in the Vicinity of the IgL Locus

Targeted integration was used to insert the GFP2 reporter at various distances from the IgL locus into chromosome 15 [International Chicken Genome Sequencing Consortium, 2004] (FIGS. 1B and 1E). FACS analysis of primary transfectants and their subclones revealed that the medians of decreased green fluorescence fell to about 3% at the +26 kb and the −15 kb positions, to 0.5% at the +52 kb position and to about 0.05% at the −135 kb position (FIGS. 1C and 1D). Although it could not be determined for the GFP2 insertions outside the IgL locus, whether the rearranged or the unrearranged chromosome was targeted, the results of the subclones were also representative for a large number of independent primary transfectants (Table 1A). Thus, hypermutation of the GFP2 reporter was detectable at insertions up to 52 kb away from the IgL locus, but incidence of mutations declined with increasing distance and were barely detectable at the −135 kb position.

Since surrounding sequences should not influence the post-transcriptional processing and translation of GFP2 transcripts, GFP2 transcription would be reflected by the green fluorescence of the cells independent of the transgene insertion site. Even for mutating transgenes, GFP2 transcription levels could be deduced from the average green fluorescence of the major cell populations which most likely expressed the non-mutated GFP sequence. As seen by FACS analysis, the average green fluorescence of the major cell populations varied slightly among the primary transfectants (FIG. 1C) most likely reflecting chromosomal position effects. However, the transfectants +52IgL^{GFP2} and IgL^{GFP2} differed more than 20 fold in their median fluorescence decreases despite similar green fluorescence of their major cell populations. This strongly suggested that the hypermutation differences among the transfectants reflected the distance of the GFP2 insertion sites to the IgL locus and not variation in GFP2 transcription.

### Example 3

### Identification of a Diversification Activator

The effects observed in Examples 1 and 2 could be explained by the presence of a cis-acting sequence that activated hypermutation in a distance-dependent manner. This putative regulatory sequence was designated Diversification Activator (DIVAC). Mapping of DIVAG was done by combining insertions of the GFP2 reporter with deletions of the IgL locus.

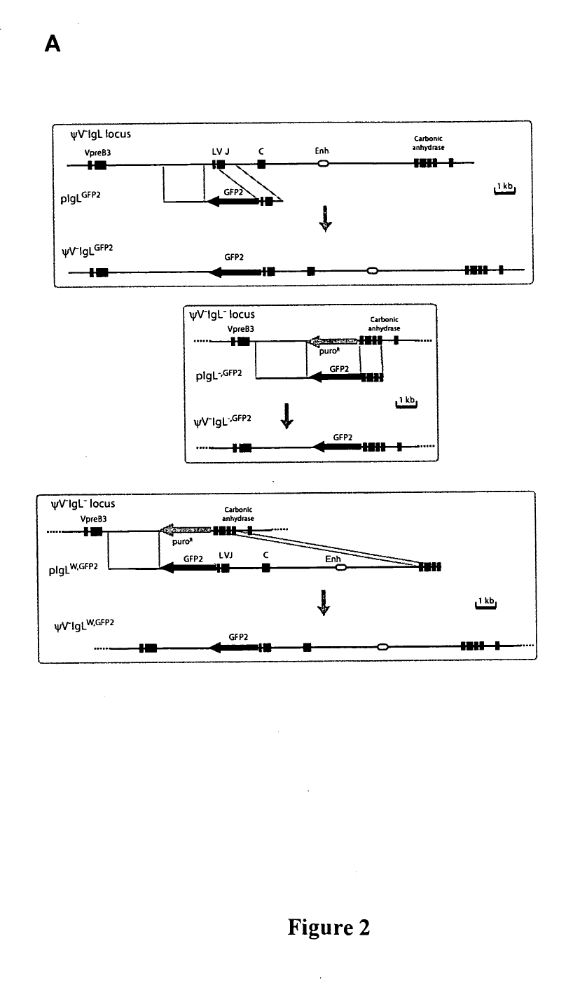

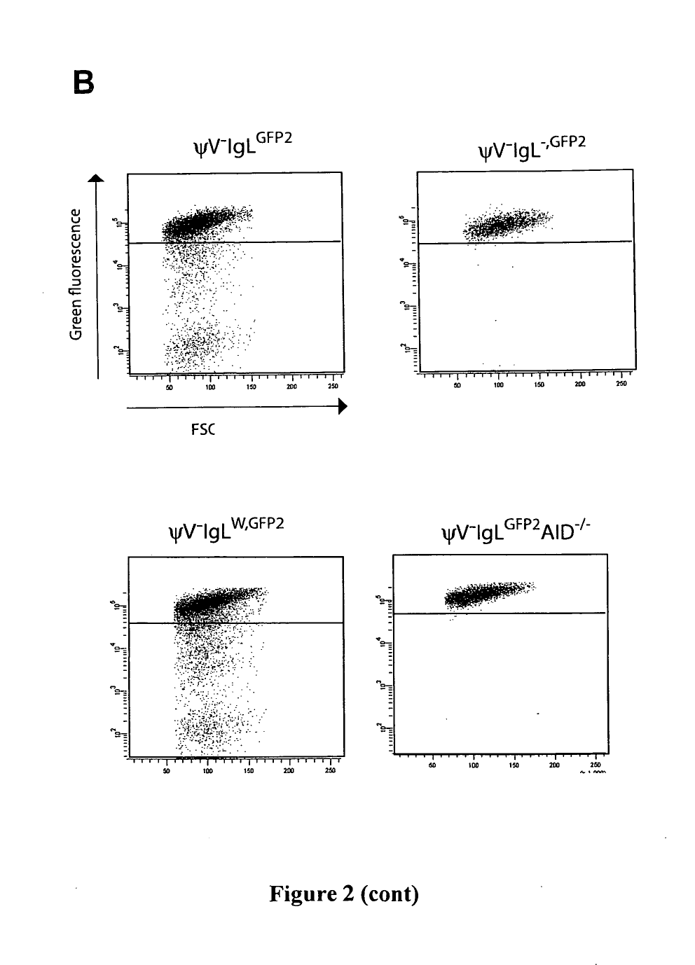

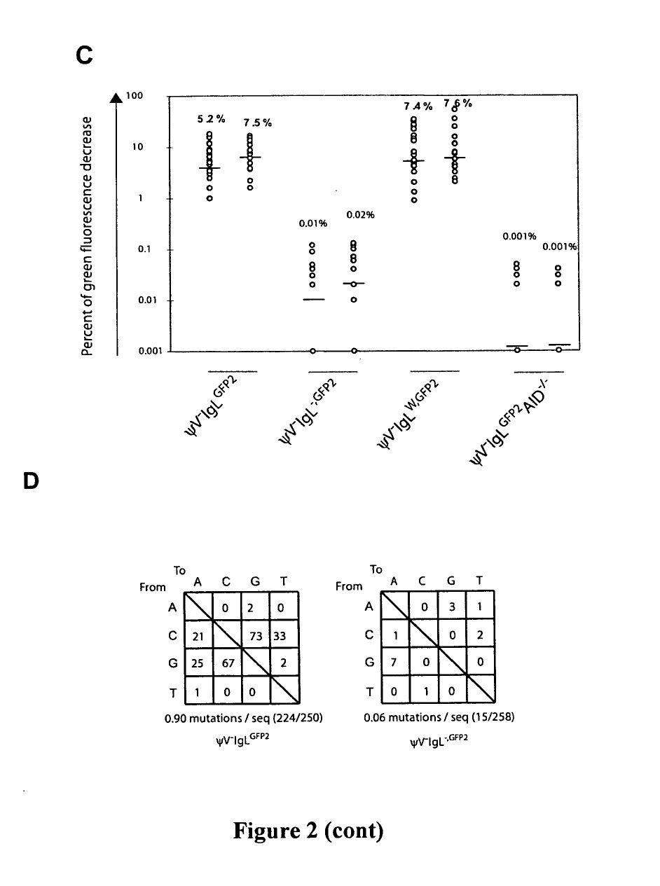

To address the role of the ψV locus, a GFP2 construct (FIG. 2A, upper part) was transfected into the clone ψV⁻AID^{R1} [Arakawa et al., 2008] in which the entire 20 kb of the ψV locus had been deleted. The transfectants ψV⁻IgL^{GFP2} expressed the GFP2 reporter at the position of the IgL promoter in the absence of the ψV locus (FIG. 2B) . FACS analysis of ψV⁻IgL^{GFP2} subclones revealed medians of 5.2% and 7.5% decreased green fluorescence (FIG. 2C), only one fold lower than the medians of the ψV positive IgL^{GFP2} subclones. As the difference between IgL^{GFP2} and ψV⁻IgL^{GFP2} subclones could be due to fluctuation effects or different AID expression levels in the AID^{R2} and ψV⁻AID^{R1} precursors, the ψV locus seems to exert little, if any stimulation on the hypermutation activity of the GFP2 reporter.

ψV⁻IgL^{GFP2} still contained a 9.8 kb fragment (referred to in the following as fragment ‘W’) of the rearranged IgL locus extending from the IgL transcription start site to the 3′ end of the carbonic anhydrase gene. To test the relevance of this fragment, a GFP2 construct was transfected into the clone ψV⁻IgL⁻ in which the entire rearranged IgL locus had been replaced by the puromycin resistance gene (P42670). The resulting transfectants ψV⁻IgL^{-,GFP2} had inserted the GFP2 reporter at the position of the deleted IgL locus (FIG. 2A, middle part and FIG. 2B). Subclones of ψV⁻IgL^{-,GFP2} showed medians of 0.01% and 0.02% decreased green fluorescence (FIG. 2C), more than 100 fold lower than the medians of ψV⁻IgL^{GFP2} subclones. This indicated that the ‘W’ fragment,—absent in ψV⁻IgL^{-,GFP2} but present in ψV⁻IgL^{GFP2}—, was required for hypermutation of the GFP2 transgene. ψV⁻IgL⁻ cells were then transfected by a construct including the GFP2 transgene and the ‘W’ fragment (FIGS. 2A, lower part). Subclones of the transfectants ψV⁻IgL^{W,GFP2} showed median green fluorescence decreases similar to the medians of ψV⁻IgL^{GFP2} subclones (FIG. 2C). Thus, the ‘W’ fragment efficiently activates hypermutation after reinsertion into the IgL locus as expected for a true DIVAC sequence.

Controls confirmed that the appearance of cells with decreased green fluorescence reflected hypermutation in the GFP2 gene. As expected, the decrease of green fluorescence in ψV⁻IgL^{GFP2} cultures depended on AID, because subclones of the AID negative transfectant ψV⁻IgL^{GFP2}AID^{-/-} showed only very low medians of 0.001% decreased green fluorescence (FIG. 2C). Furthermore, GFP open reading frames amplified from ψV⁻IgL^{GFP2} cells six weeks after subcloning showed 0.9 nucleotide substitutions on average (FIG. 2D). The most prevalent mutations were C to G and G to C transversions as previously observed for hypermutation of the IgL VJ segments from ψV deleted clones [Arakawa et al., 2004]. In contrast, only a very low number of nucleotide substitutions, were found in the GFP gene of ψV⁻IgL^{-,GFP2} cells (FIG. 2D).

### Example 4

### Mapping of DIVAC

Mapping of the ‘W’ Fragment

A new series of targeting constructs was transfected into ψV⁻IgL⁻ to characterize the ‘W’ fragment by step-wise deletions (FIG. 3A). FACS analysis of subclones from the different transfectants showed a variable but progressive loss of hypermutation activity when the ‘W’ fragment was shortened from either end (FIG. 3C). The 4 kb ‘S’ fragment in the middle of the ‘W’ fragment which included the previously identified IgL enhancer [Bulfone_Paus et al., 1995] still produced median green fluorescence decreases of 2.7% and 1.7%. In contrast, the upstream ‘B’ and the downstream ‘P’ fragments on their own produced median green fluorescence decreases of about 0.13% and 0.05% respectively, which are low in absolute terms, but clearly above the medians of ψV⁻IgL^{-,GFP2}. If either one of these fragments was combined with the ‘S’ fragment in the ‘F’ and ‘K’ fragments respectively, the median decreases of green fluorescence were elevated about 3 times. This suggested that the DIVAC of the chicken IgL locus consisted of a central core region and partially redundant flanking regions which contributed to the overall activity.

The average green fluorescence in the main population of ψV⁻IgL^{W,GFP2} was increased compared to ψV⁻IgL^{-,GFP2} (FIG. 2B) perhaps due to the additional stimulation of the RSV promoter in GFP2 by the IgL enhancer of the ‘W’ fragment. However, the relatively small decrease of GFP2 transcription seen in ψV⁻IgL^{-,GFP2} was unlikely to be responsible for the more than 300 fold reduction of hypermutation. Analysis of the ‘W’ fragment deletions also strongly argued against the possibility that differences in hypermutation were caused by alterations of GFP2 transcription since primary transfectants of all fragments shown in FIG. 3B showed similar GFP transcription levels.

Mapping of the ‘S’ Fragment

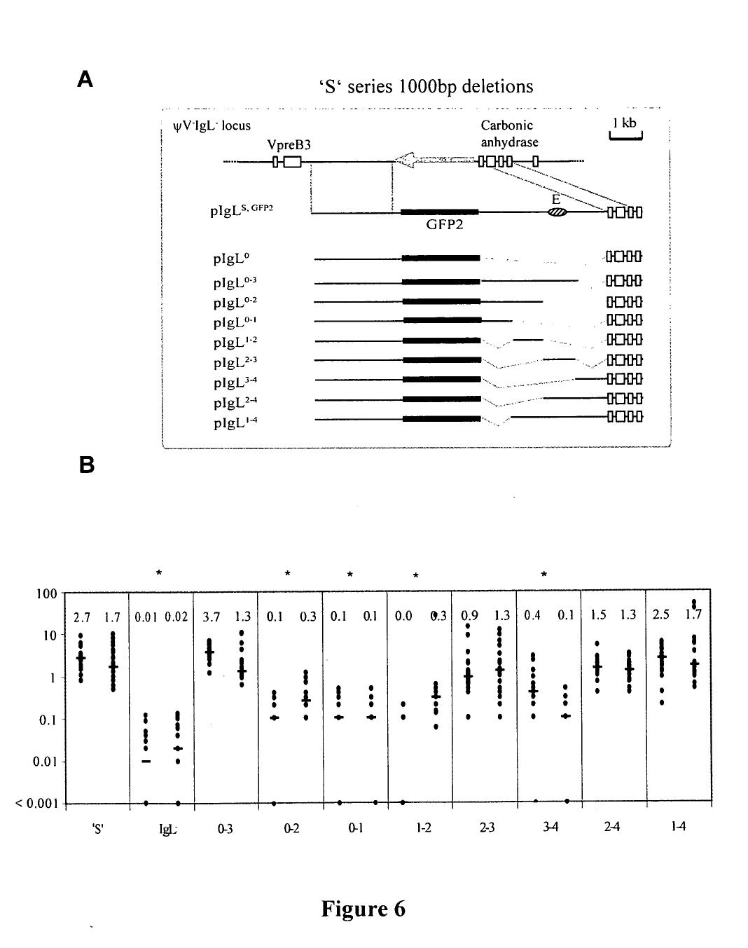

As described supra, an approximately 4 kb segment of the rearranged DT40 IgL locus (‘S’ fragment) was essential for AID-mediated hypermutation. The DNA fragment spans 2 kb sequence upstream and 1.6 kb sequence downstream of the already identified enhancer. To locate an active core (or cores) of the ‘S’ fragment which might serve as platform for the assembly of a putative hypermutation machinery and is locating AID to the Ig locus, a detailed deletion analysis of the 4 kb region was conducted. The 5′ starting point of the ‘S’ fragment (further referred to as ‘0-4’ fragment) was defined as zero point and the sequence was divided into four 1 kb regions (FIG. 6). A series of 1 kb step-wise deletions was performed, starting from the 5′ as well as from the 3′ end of the sequence.

The ‘0-4’ fragment itself produced a median decrease of green fluorescence of 2.7% and 1.7%. The median green fluorescence decrees of the deletion constructs was compared to the ‘0-4’ control and p-values were calculated using Mann-Whitney U-test. Only values of p<0.001 were accepted as significant.

Deletions starting from the 3′ end showed that the last 1 kb region (‘3-4’) had no significant relevance for AID recruitment, since the median green fluorescence decrease of the ‘0-3’ fragment was still 3.7% and 1.7%. Deleting an additional 1 kb from the 3′ end (‘0-2’) clearly reduced mutations to 0.1% and 0.3%, suggesting that ‘2-3’, which includes the complete enhancer sequence, plays a role in the hypermutation process. Deletions starting from the 5′ end supported this theory. The fragment ‘2-4’ ranged with 1.5% and 1.3% in the same level as ‘0-4’, but with removing ‘2-3’, the median GFP mutation level of ‘3-4’ dropped to significantly decreased values of 0.4% and 0.1%. Accordingly, deletion from both ends of the ‘0-4’ fragment supported a role of ‘2-3’ for AID recruitment.

Examination of the 1 kb fragment ‘2-3’ confirmed the result, as it was the only 1 kb fragment with a hypermutation enhancing effect (0.9% and 1.3%). Neither of ‘0-1’ (0.1% both), ‘1-2’ (0% and 0.3%) and ‘3-4’ initiated hypermutation to a significant degree although diversification activity in these clones was still above the background level if compared to the IgL This result spoke against an additive effect (i.e., an enhancing effect on their own) of ‘0-1’, ‘1-2’ and ‘3-4’ on hypermutation. However, there could be some cumulative effect of these fragments, as hypermutation level increases slightly if one of the fragments was added to ‘2-3’. The ‘2-3’ fragment appears to include the core cis-element responsible for targeting the hypermutation. The results also indicated an important role of the enhancer, which resides within ‘2-3’ in the hypermutation activation process.

Fine Mapping of the ‘0-4’ Fragment

A deletion analysis of internal deletions in ‘0-4’ is useful in defining a smaller active fragment. At the same time, it would help to identify single acting sequences or elements which act in cooperative way, even if they are distant from each other. A drop in the somatic hypermutation frequency would indicate that an essential element involved in AID-mediated diversification is deleted.

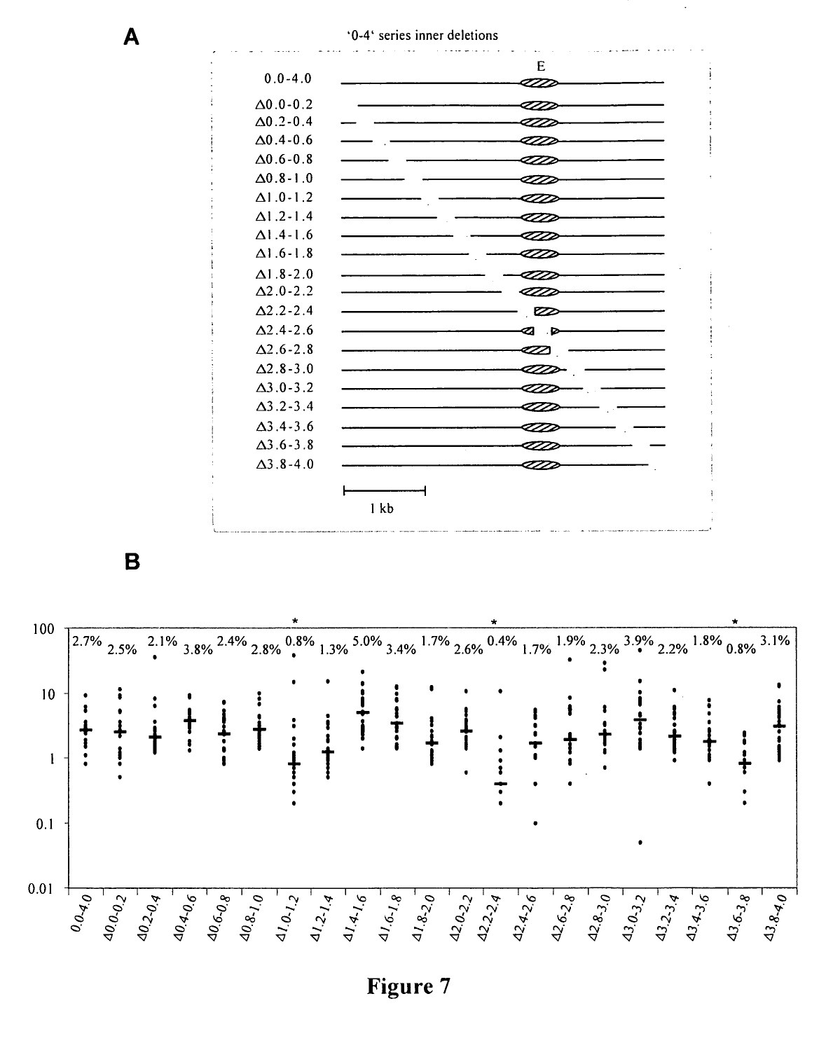

Fine mapping of the ‘0-4’ fragment was performed with help of a series of 200 bp deletions (FIG. 7). The deletion clones were compared to the original ‘0-4’ clone and the significance was calculated according to the Mann-Whitney U-test. Somatic hypermutation activity from three out of 20 deletion clones was significantly reduced. The constructs ‘0.0-1.0/1.2-4.0’ and ‘0.0-3.6/3.8-4.0’ showed a decreased GFP expression of 0.8% and the construct ‘0.0-2.2/2.4-4.0’ a decreased GFP expression of 0.4%. To exclude an experimental artifact of these three constructs, the results were confirmed by screening one additional clone for each construct. The decreased GFP expression for the second clone of ‘0.0-1.0/1.2-4.0’ and ‘0.0-3.6/3.8-4.0’ was 3.5% and 4.9% respectively (data not shown). The clonal difference could be due to a loss or inhibition of the AID cDNA expression cassette or other genes involved in somatic hypermutation, as random integration of the transfected constructs could not be controlled. A second clone of ‘0-2.2/2.4-4.0’ showed a significantly decreased GFP expression with 0.5% (data not shown), thus confirming the data obtained with the first clone. The identified 200 bp region ‘2.2-2.4’ is part of the previously identified ‘2-3’ fragment and is located within the IgL enhancer. These results support a role of the enhancer for AID recruitment to the hypermutating locus.

Mapping of the ‘2-3’ Fragment

As typical protein binding sites are in range of 10-20 bp, a more detailed deletion analysis was performed in the ‘2-3’ fragment to define specific protein binding regions. A combination of 50 bp internal deletions and 50 bp serial deletions from both ends is useful to identify redundant motifs or a repetition of motifs.

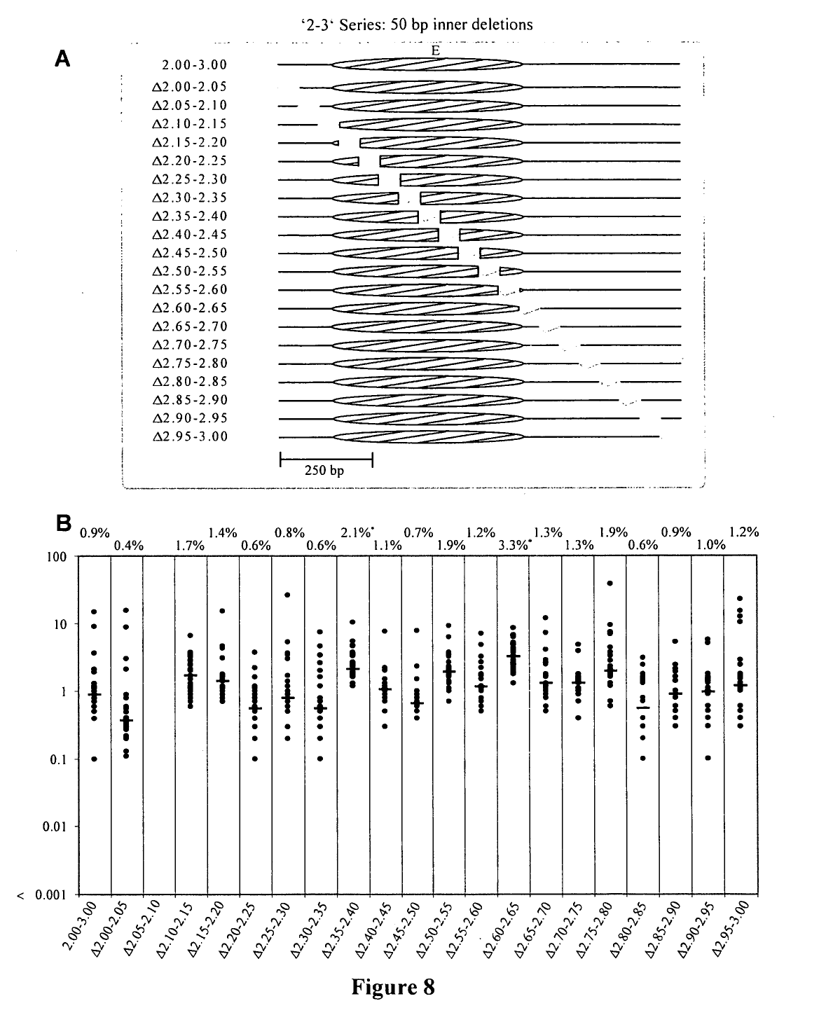

Mapping of the ‘2-3’ fragment was performed by analyzing 50 bp end and internal deletions of the ‘2-3’ fragment (FIGS. 8, 9 and 10).

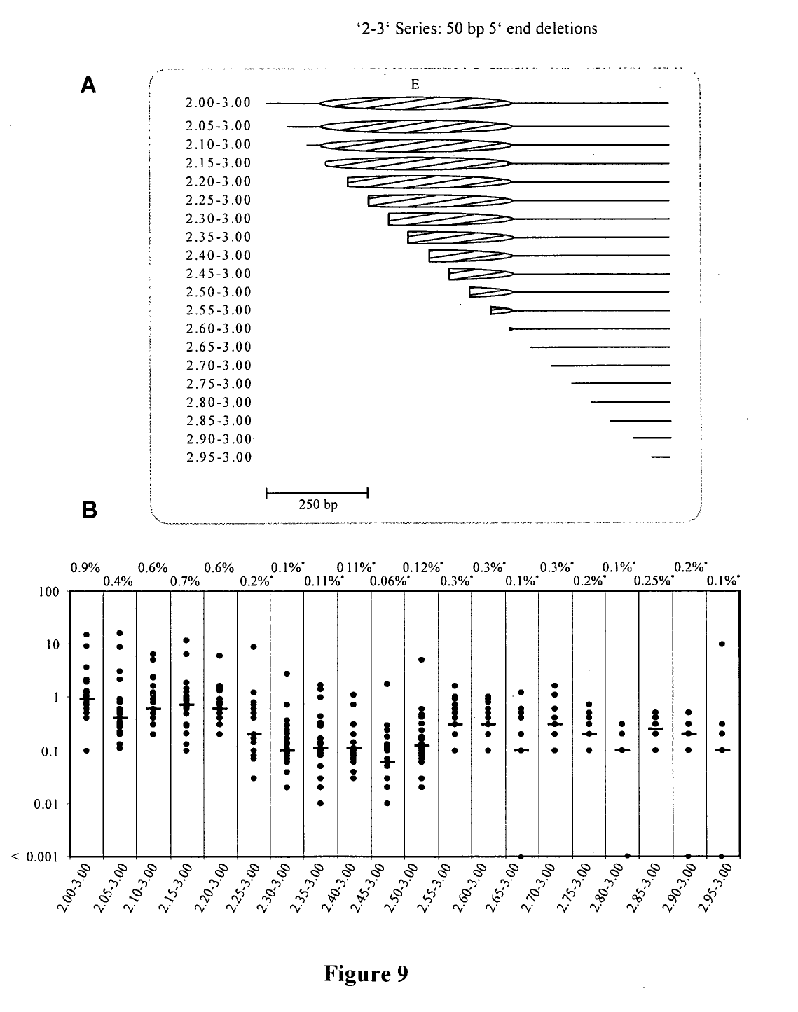

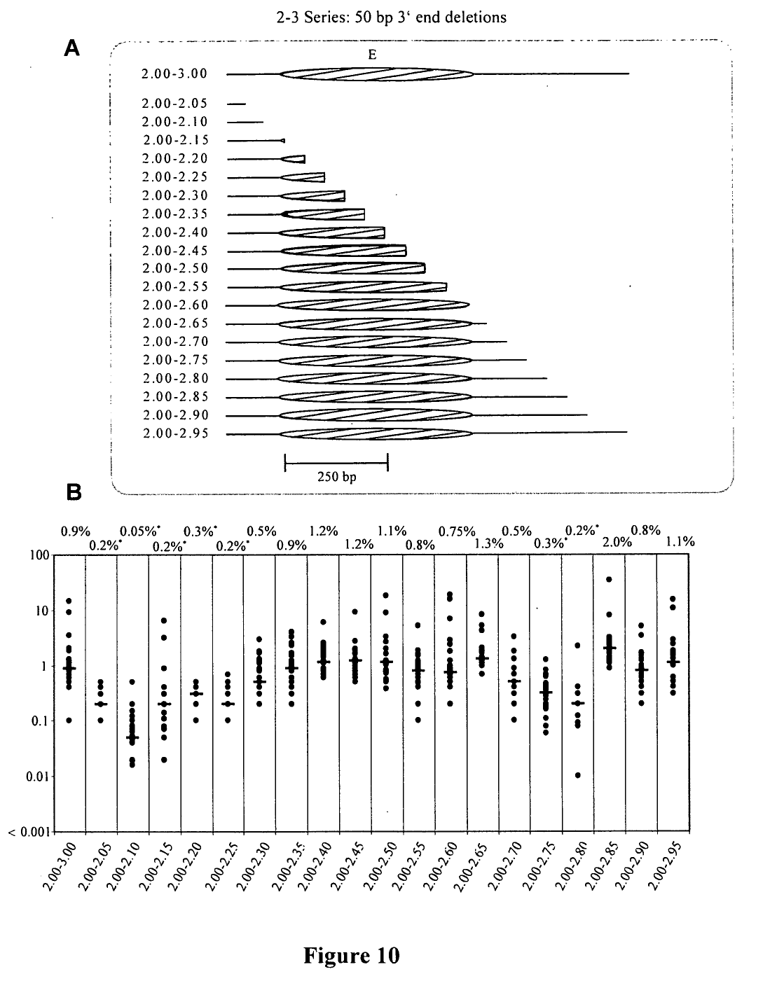

The 50 bp progressively increasing deletion series starting from the 5′ end displayed a drop of GFP expression from ‘2.2-3.0’ with 0.6% to ‘2.25-3.0’ with 0.2% (FIG. 9). Removal of the ‘2.2-2.25’ confirms the importance of the ‘2.2-2.4’ fragment. However, as an internal deletion of ‘2.2-2.25’ had no effect (FIG. 8), it is expected that redundant elements are present in the ‘2.2-2.4’ region. Increasing 50 bp deletions at the 3′ end indicated the presence of an additional element relevant for the hypermutation activity of the ‘2.2-2.4’ fragment in ‘2.25-2.3’ (FIG. 10). Thus, the GFP mutation level rose from 0.2% for ‘2-2.25’ to 0.5% for ‘2-2.3’. Therefore, several different or repetitive motifs in the ‘2.2-2.3’ region were expected. However, the identified motifs in ‘2.2-2.25’ and ‘2.25-2.3’ appear to require additional elements for proper function, as an internal deletion of ‘2.2-2.25’ and ‘2.25-2.3’ (FIG. 8) showed no significant loss of hypermutation activity.

In the 3′ end deletion series, a drop of GFP decrease associated with ‘2-2.75’ and ‘2-2.8’ could additionally be detected (FIG. 10). This might reflect the influence of a silencing element. However, a deletion of this region in the 50 bp internal deletion series was unaffected (FIG. 8). The addition of ‘2.8-2.85’, could compensate the effect. These results confirm the role of the 200 bp element ‘2.2-2.4’ as hypermutation activator.

Thus, the 50 bp internal deletions showed no significant reduction of GFP expression. As random deletions were produced, it is tempting to speculate that only parts of sequence motifs were deleted, without deleting the conserved parts. Additionally, the ‘2-3’ fragment might contain redundant elements which could substitute for each other.

Reconstitution and multimerization of the ‘2.2-2.4’ fragment Deleting ‘2.2-2.4’ in the ‘0-4’ fragment led to a five-fold reduction of decreased GFP expression. To find out if ‘2.2-2.4’, similar to ‘2-3’, was not only necessary but also sufficient for hypermutation, the ‘2.2-2.4’ fragment was reconstituted in ψV⁻IgL⁻. A median decrease of GFP expression of 0.6% could be detected. This result confirmed that ‘2.2-2.4’ contained one or several elements sufficient for hypermutation. However, the number of GFP negative cells was lower than in ‘2-3’ (0.9%, 1.3%) and significantly lower than in ‘0-4’ (2.7%, 1.7%).

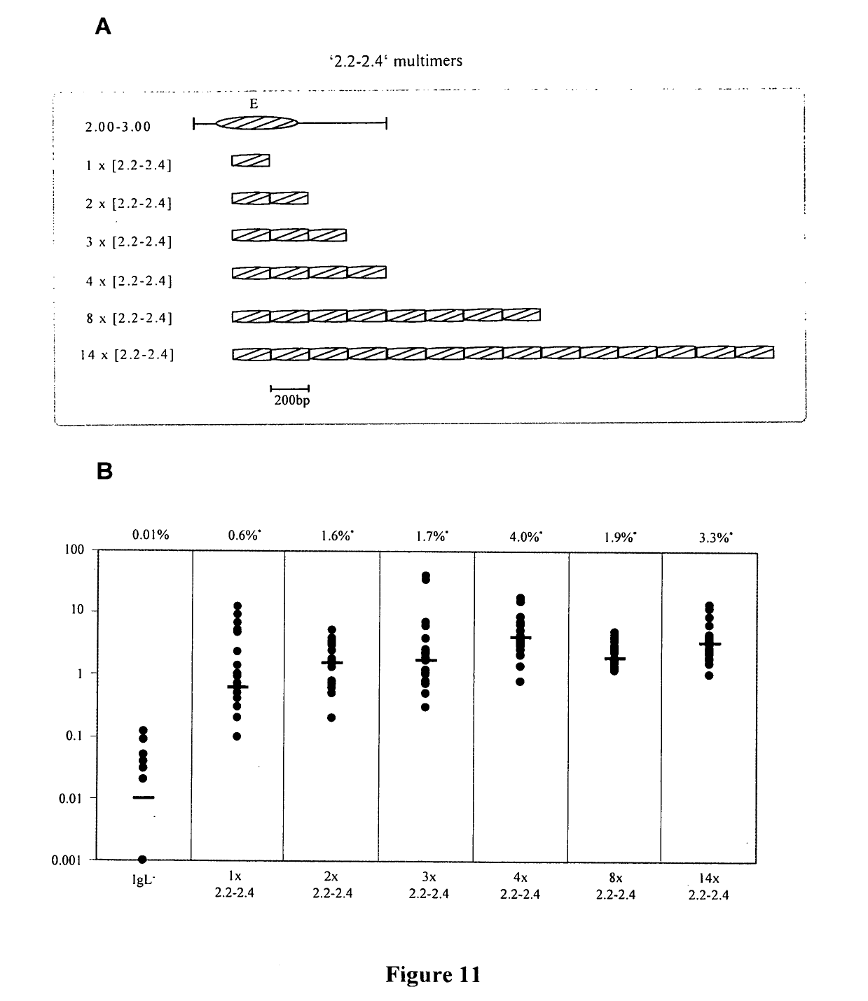

To find out whether this effect was due to additive elements or to repetitive motives, the ‘2.2-2.4’ fragment was multimerized (FIG. 11). Doubling the element enhanced the median decrease of fluorescence by two-fold (1.55%), whereas quadruplicating the element resulted in the fraction of GFP negative cells reaching a level even higher than that of ‘0-4’ (4%). Multimerizing the sequence 8 times and 14 times failed to further increase the hypermutation rate. It is possible that there is a saturation level for the ‘2.2-2.4’ fragment, and absence of other motifs and/or intrinsic factors can limit the process. These data confirms that the ‘2.2-2.4’ fragment is necessary and sufficient for inducing hypermutation.

### Example 5

### Hypermutation at Non-Ig Loci

‘W’ (9.8 kb) Fragment

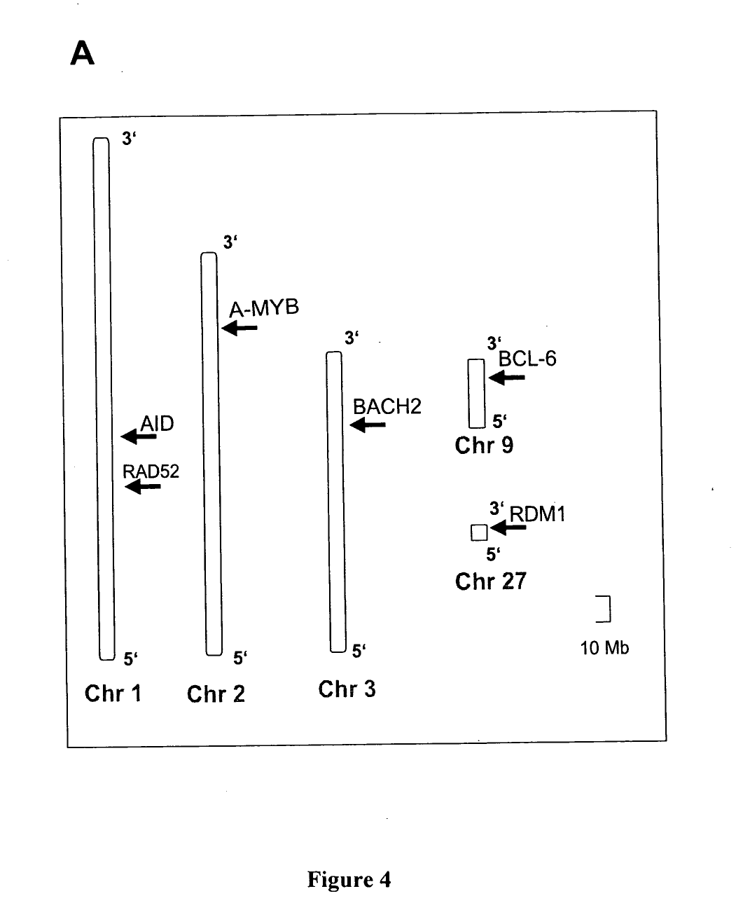

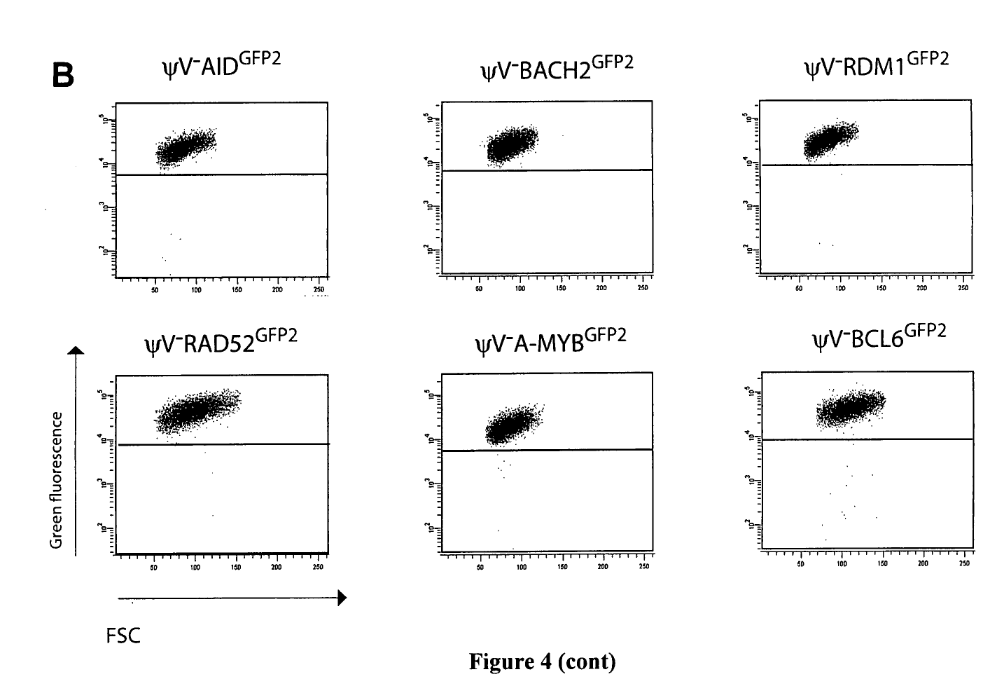

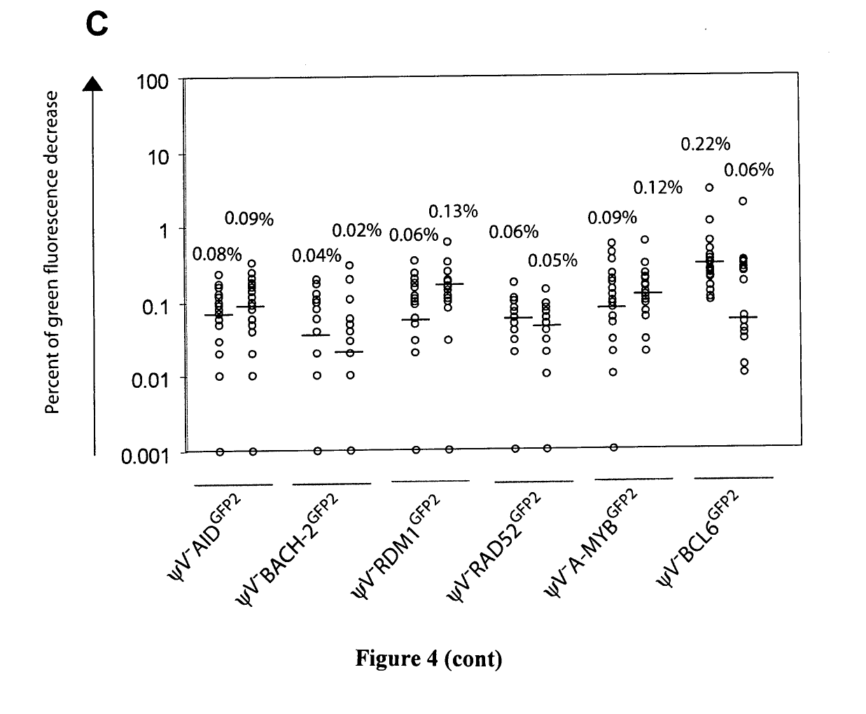

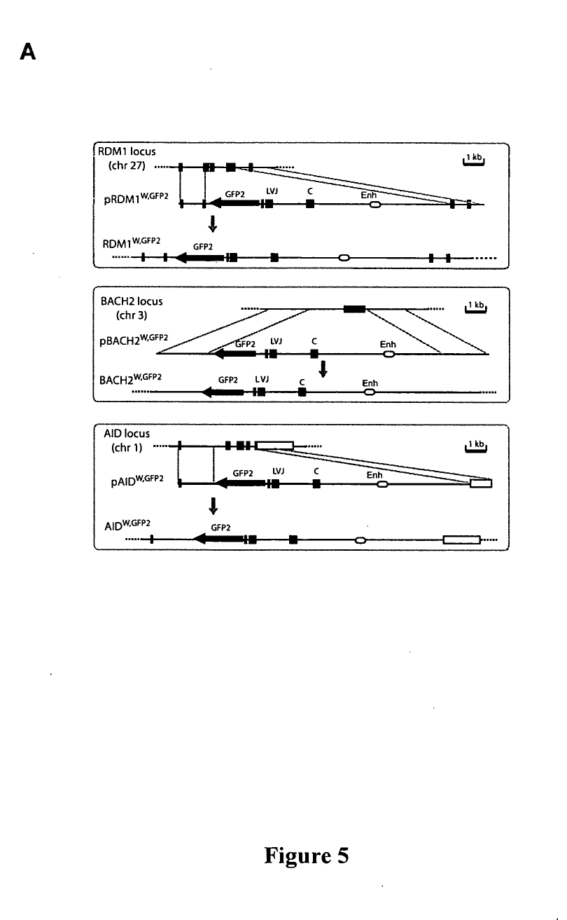

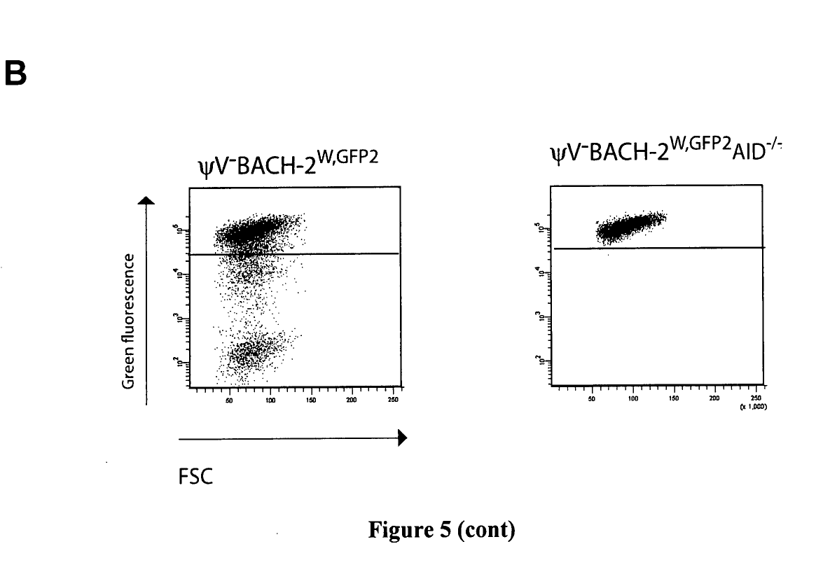

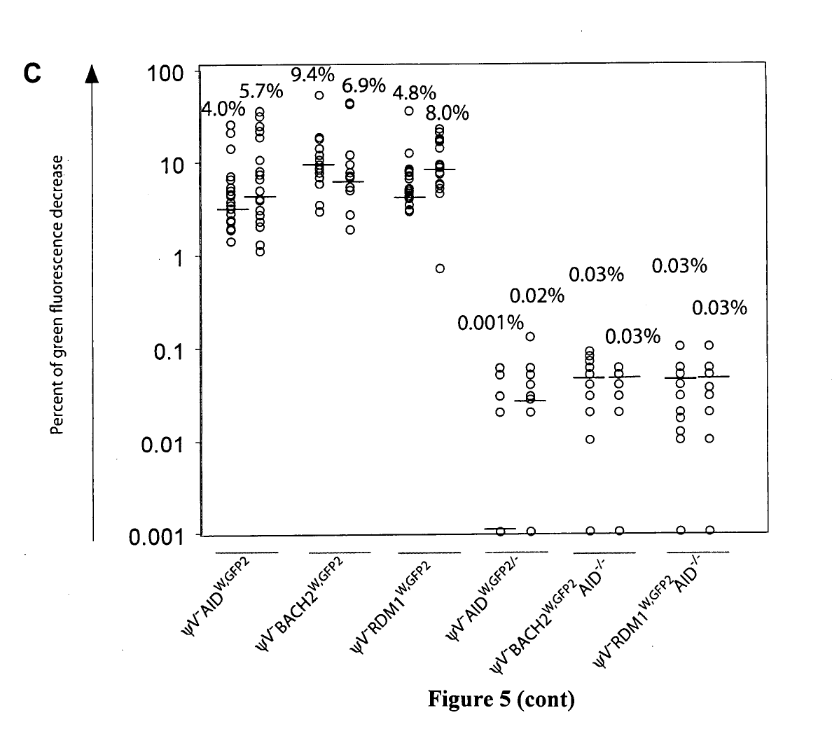

To confirm that the GFP2 reporter on its own is stably expressed at non-Ig loci, six loci on five different chromosomes [International Chicken Genome Sequencing Consortium, 2004] were targeted by transfection of GFP2 constructs into ψV⁻AID^{R1} (FIGS. 4A and 1F). Neither the primary transfectants (FIG. 4B, Table 1B) nor their subclones (FIG. 4C) showed high percentages of decreased green fluorescence. Depending on the experiment and the insertion site, the medians of the subclones ranged from 0.02% to 0.22% indicating that the mutation rates of the GFP2 reporter at the chosen loci were 50 to 500 fold lower than at the IgL locus. However, these medians were about 2-10 fold higher than the medians of various subclones from AID negative transfectants (FIG. 2C and 5C) confirming a slight increase in the background mutation rates in AID expressing B cells [Liu et al., 2008].

GFP2 was then inserted together with the ‘W’ fragment into the respective AID, BACH2 (NC_{—}006090) and RDM1 (BAC02561) loci of ψV⁻AID^{R1} (FIGS. 5A and 5B). Subclones of the transfectants ψV⁻AID^{W,GFP2}, ψV⁻BACH2^{W,GFP2} and ψV⁻RDM1^{W,GFP2} showed high median decreases of green fluorescence between 4.0% and 9.4% (FIG. 5C) similar to the medians for ψV⁻IgL^{GFP2} and ψV⁻IgL^{R,GFP2} subclones. GFP2 hypermutation was AID dependent, since subclones of the AID negative transfectants ψV⁻AID^{W,GFP2/-}, ψV⁻BACH2^{W,GFP2/-}AID^{-/-} and ψV⁻RDM1^{W,GFP2/-}AID^{-/-} showed very low medians of decreased green fluorescence in the range of 0.001% to 0.03%. These results demonstrated that the ‘W’ fragment was able to activate AID mediated hypermutation at loci which otherwise did not support hypermutation.

‘2.2-2.4’ Fragment

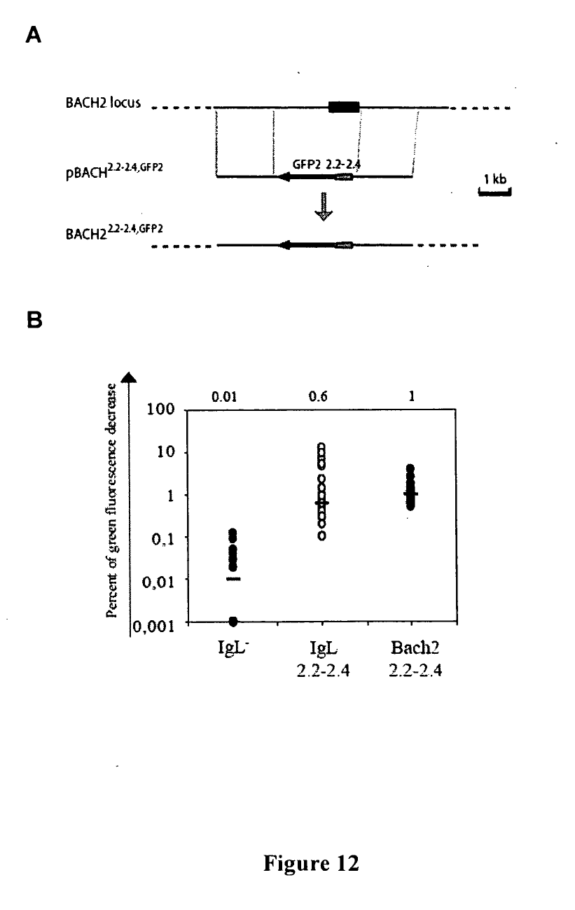

The mapping of the DIVAC sequence indicated that ‘2.2-2.4’ acted like a true hypermutation core element (“HyCorE”) that is capable of starting hypermutation. Multimerization of this element had an additive effect. The ability of ‘2.2-2.4’ to target hypermutation to a specific, non-Ig locus was tested. In analogy with the experiments described in this example for the ‘W’ fragment in different non-Ig loci, hypermutation activity of the ‘2.2-2.4’ fragment was tested in the BACH2 locus (FIG. 12). The GFP2 reporter together with ‘2.2.-2.4’ was targeted to the BACH2 locus. The resulting clone AID^{R1}IgL⁻BACH2^{+/2.2-2.4,GFP2} was subcloned and analyzed by FACS. The median of decreased green fluorescence was compared to that of the clone AID^{R1}IgL^{2.2-2.4,GFP2}.

Insertion of the GFP2 reporter alone at the position of the BACH2 locus of ψV⁻AID^{R1} (AID^{R1}IgL⁻BACH2^{+/GFP2}) did not lead to mutations in the GFP transgene. When combined with the ‘2.2-2.4’ fragment, however, GFP2 became a target for mutations with a median of about 1% decrease in green fluorescence. This is somewhat higher than in “IgL 2.2-2.4”, where the fragment was inserted at the position of the deleted IgL locus. At the same time, both values were significantly different from “IgL−”, a cell line in which the GFP2 reporter is not hypermutating due to the deletion of the entire IgL locus.

These results demonstrate that the ‘2.2-2.4’ fragment is able to trigger hypermutation at non-Ig loci, indicating that it is both necessary and sufficient for activating hypermutation.

### Example 6

### Homologues of the ‘2.2-2.4’ Fragment in Duck and Turkey

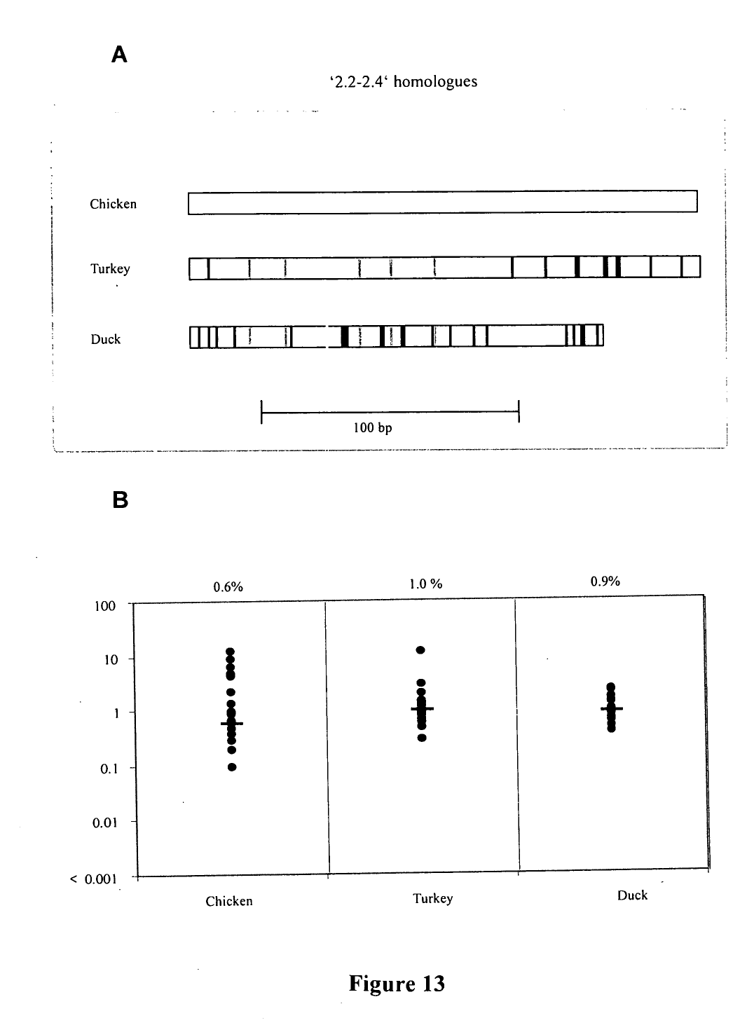

Homologous of the ‘2.2-2.4’ fragment in duck and turkey were cloned and sequenced. Sequence homology between the three species is very high, with the homology of the duck sequence of 85%, and that of the turkey sequence of 92% (FIG. 13). To demonstrate functional conservation, the duck (SEQ ID NO:116) and turkey (SEQ ID NO:115) ‘2.2-2.4’ fragments were inserted into the ψV⁻IgL⁻ cell line. The duck sequence resulted in a GFP expression decrease of 0.9% and the turkey sequence in a decrease of 1.0%. This indicates that the diversification activator sequence is conserved through different species.

### Example 7

### Analysis of Sequence Motifs

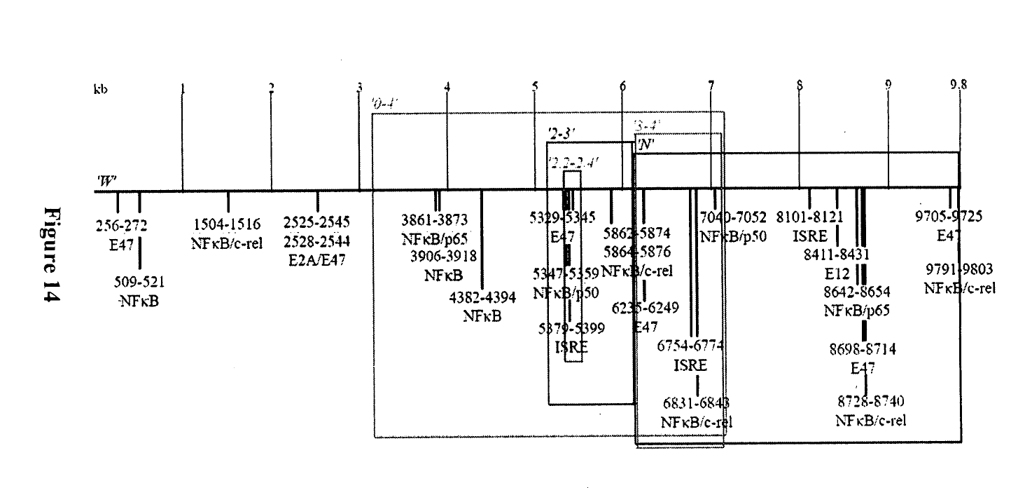

A bioinformatical analysis of the about 9.8 kb ‘W’ fragment was performed with the purpose of identifying positions of important protein binding motifs. A motif analysis of the 9.8 kb fragment, considering the motifs E-Box, NF-kB and ISRE is illustrated in FIG. 14. These motifs were chosen because they are the most interesting candidates within the ‘2.2-2.4’ fragment. In particular, NEMO, NK-kB Essential Modulator, a factor activating NF-kB is known to influence hypermutation and ISRE, Interferon-Stimulated Response Element, which binds interferon regulatory factors, is known to interact with NF-kB [Jain et al., 2004; Souto-Carneiro et al., 2008; Naschberger et al., 2004; Wu et al., 1994; MatInspector transcription factor database (www.genomatix.de; Quandt et al., 1995; Cartharius et al., 2005)].

FIG. 14 shows that the tightest cluster of the three motifs is found within the ‘2.2-2.4’ fragment (FIG. 14, orange box). The motifs present in the ‘2.2-2.4’ fragment are also contained in the ‘9.6-9.8’ fragment. Another cluster is within the sequence of about 1 kb between 6-7.1 kb (FIG. 14, light blue box). This part of the sequence is contained within ‘0-4’ and ‘N’, i.e., both fragments with hypermutating activity. In fact, this 1 kb fragment is the only difference between the ‘N’ and the ‘P’ fragments and it enhances hypermutation 100-fold (see FIG. 3). One more cluster is at 8-9 kb and is part of ‘N’. It is possible that the cluster in ‘3-4’ interacts with the cluster between 8 kb and 9 kb. In the first 5 kb of the sequence, which do not have a significant hypermutating activity, no clustering of the three motifs is present.

The coordinates of the deletion fragments on the 9.8 kb scale are indicated in Table 3.

TABLE 3
Position of fragments indicated in FIG. 14 within
the ‘W’ fragment

| | | | Location within ‘W’ |
|---|---|---|---|
| | | | (SEQ ID NO: 1) |
| ‘2-3’: | Forward: | GAAGCTAGCCACGACAGCTGGGGCCACACAAAGA | 5114-6106 |
| | Reverse: | GAAACTAGTAAGCTCAGGGTCTCAGTTTGGAGCT | |
| ‘3-4’: | Forward: | GAAGCTAGCGTCACAGGTTGTAACAGGCTGACAT | 6107-7099 |
| | Reverse: | GAAACTAGTATTGCTGCAGTGCAAACGCCCTGGT | |
| ‘0-4’: | Forward: | GAAGCTAGCTTTATGCTGGGAACAGGGGGAGTTC | 3098-7099 |
| or ‘S’ | Reverse: | GAAACTAGTATTGCTGCAGTGCAAACGCCCTGGT | |
| ‘N’: | Forward: | GAAGCTAGCGTCACAGGTTGTAACAGGCTGACAT | 6107-9784 |
| | Reverse: | GAAGCTAGCATGGGATGGAAGGGCCCGTCTGGCC | |
| ‘9.6-9.8’ | | | 9509-9802 |

It is thus likely that the ‘W’ fragment is composed of multiple redundant motifs with high similarity to that of ‘2.2-2.4’. Results of multimerization (Example 4; FIG. 11) militate in favor of this hypothesis. Moreover, these motifs cluster especially in those parts of the ‘W’ sequence which were proven to be relevant for the induction of hypermutation.

The analysis supports of a theory according to which the “hypermutation motif” has an A-B-B-A structure. The first 800 bp of the 9.8 kb fragment (2825-3625) correspond to motif A. Mofif A is a motif, which is an enhancer of a core motif, or motif B. Motif B is for example, the ‘2.2-2.4’ fragment. The two core elements (B) have definite motif homology as do the two enhancing elements (A). It is known that defects in NEMO stop the process even with AID present. This points to a very definite activating core element where signal transduction is interpreted into somatic hypermutation, gene conversion and class switch recombination activity. While the elements listed above are likely the main ones, possible enhancing motif factors such as SP1, AP1, E2A/Thing1, PAX 2/4/5 family may also play a role.

### Example 8

### Materials and Methods

Targeting constructs. The GFP2 construct was made by combining the RSV promoter—GFP open reading frame of pHypermut2 [20] with a PCR amplicon including an IRES [Arakawa et al., 2004], the blasticidin resistance gene and the SV40 polyadenylation signal [Arakawa et al., 2001]. The PCR was performed using the primers described in the Supplementary table 2. GFP2 was flanked by unique BamHI restriction sites for easy cloning into the targeting vectors.

All targeting constructs except the ones belonging to the series of ‘W’ fragment deletions and reconstitutions were made by cloning the arms sequences into pBluescriptKS+ (Stratagene, CA) and then inserting GFP2 either into unique BamHI or BglII sites as shown in FIG. 5A and FIGS. 1E and 1F. Targeting of AID [Arakawa et al., 2001] and RDM1 [Hamimes et al., 2004] have been previously described. The PCR amplifications of all target arms were performed using the Expand long template PCR System (Roche, Switzerland), DT40 genomic DNA as template and primers as described in Table 2.

Since the ‘W’ fragment was difficult to amplify as a single sequence, it was sequentially cloned by combining upstream and downstream PCR amplicons with a 2.2 kb AvrII/SpeI restriction fragment excised from the rearranged IgL targeting construct ‘Construct R’ [Buerstedde and Takeda, 1991]. The sequence of the AvrII/SpeI restriction fragment is A/T rich and localized between the J segment and the C region. The assembled ‘W’ fragment was sequenced and deposited into Genbank under the accession number bankit FJ482234. Constructs belonging to ‘W’ fragment deletion series were made by cloning GFP2 between the target arms and then inserting the ‘W’ fragment or parts thereof into unique NheI/SpeI sites. A BamHI fragment containing GFP2 and the ‘W’ fragment was incorporated into the AID, BACH2 and RDM1 targeting vectors to test the activity of the ‘W’ fragment in non-Ig loci.

Cell culture. Cells were cultured in chicken medium (RPMI-1640 or DMEM/F-12 with 10% fetal bovine serum, 1% chicken serum, 2 mM L-glutamine, 0.1 μM β-mercaptoethanol and penicillin/streptomycin) at 41° C. with 5% CO2. Transfections were performed by electroporation, using 40 μg of linearized plasmid DNA with a Gene Pulser Xceil (BIO-RAD) at 25 μF and 700 V. Stable transfectants were selected by culturing in 15 μg/ml of blasticidin. Transfectants having integrated the transgenic constructs by targeted integration were identified by PCR using an inside primer from the SV40 polyadenylation signal sequence of GFP2 together with a primer derived from the sequence outside the target arm (Table 2). In case of insertions into the IgL locus, targeted integration into the rearranged allele was verified by amplifying the VJ intervening sequence of the unrearranged locus. The AID reconstituted clone AID^{R2} was generated from the AID deleted clone AID^{-/-} [Arakawa et al., 2002] by transfection of a construct which targeted an AID cDNA expression cassette into one of the deleted AID loci. The AID negative transfectants were produced by transfecting ψV⁻AID^{-/-} [Arakawa et al., 2004].

Flow cytometry. The phenotype of each mutation was determined by FACS analysis of at least two independent targeted transfectants and twenty-four subclones of each. The primary transfectants were analyzed by FACS about three weeks after transfection and the subclones two weeks after subcloning. As the green fluorescence levels in the main populations varied slightly among the transfectants, the gates to separate the main population of green fluorescent cells from cells showing decreased or lost green fluorescence were adapted accordingly. At least 50% events falling into the live cell gate were collected for each primary transfectant or subclone. Subclones in which more than 50% of the live cell events fell into the gates for decreased or lost green fluorescence were excluded from the analysis as they might represent the expansion of a precursor cell already expressing a mutated GFP2 transgene at the time of subcloning.

GFP Gene Sequencing. To minimize PCR-introduced mutations, Pfu Ultra hotstart polymerase (Stratagene) was used for the amplifications of the GFP open reading frames prior to sequencing. Sequencing and sequence analysis were performed as previously described [Arakawa et al., 2004].

### REFERENCES

1. Muramatsu M, Kinoshita K, Fagarasan S, Yamada S, Shinkai Y, et al. (2000) Class switch recombination and hypermutation require activation-induced cytidine deaminase (AID), a potential RNA editing enzyme. Cell 102: 553-563.
2. Arakawa H, Hauschild J, Buerstedde J M (2002) Requirement of the activation-induced deaminase (AID) gene for immunoglobulin gene conversion. Science 295: 1301-1306.
3. Harris R S, Sale J E, Petersen-Mahrt S K, Neuberger M S (2002) AID is essential for immunoglobulin V gene conversion in a cultured B cell line. Curr Biol. 12(5):435-8.
4.Di Noia J, Neuberger M S (2002) Altering the pathway of immunoglobulin hypermutation by inhibiting uracil-DNA glycosylase. Nature 419: 43-48.
5. Rada C, Di Noia J M, Neuberger M S (2004) Mismatch recognition and uracil excision provide complementary paths to both Ig switching and the A/T-focused phase of somatic mutation. Mol Cell. 16(2):163-71.
6. Peters A, Storb U (1996) Somatic hypermutation of immunoglobulin genes is linked to transcription initiation. Immunity. 4(1):57-65.
7. Shinkura R, Tian M, Smith M, Chua K, Fujiwara Y, et al. (2003) The influence of transcriptional orientation on endogenous switch region function. Nat Immunol. 4(5):435-41.
8. Shen H M, Peters A, Baron B, Zhu X, Storb U (1998) Mutation of BCL-6 gene in normal B cells by the process of somatic hypermutation of Ig genes. Science. 280(5370):1750-2.
9. Liu M, Duke J L, Richter D J, Vinuesa C G, Goodnow C C, et al. (2008) Two levels of protection for the B cell genome during somatic hypermutation. Nature. 451(7180):841-5.
10. Odegard V H, Schatz D G (2006) Targeting of somatic hypermutation. Nat Rev Immunol. 6(8):573-83.
11. Betz A G, Milstein C, González-Fernández A, Pannell R, Larson T, et al. (1994) Elements regulating somatic hypermutation of an immunoglobulin kappa gene: critical role for the intron enhancer/matrix attachment region. Cell. 77(2):239-48.
12. Klotz E L, Storb U (1996) Somatic hypermutation of a lambda 2 transgene under the control of the lambda enhancer or the heavy chain intron enhancer. J Immunol. 157(10):4458-63.
13. Klix N, Jolly C J, Davies S L, Brüggemann M, Williams G T, et al. (1998) Multiple sequences from downstream of the J kappa cluster can combine to recruit somatic hypermutation to a heterologous, upstream mutation domain. Eur J Immunol. 28(1):317-26.
14. Kong Q, Zhao L, Subbaiah S, Maizels N (1998) A lambda 3′ enhancer drives active and untemplated somatic hypermutation of a lambda 1 transgene. J Immunol. 161(1):294-301.
15. van der Stoep N, Gorman J R, Alt F W (1998) Reevaluation of 3′Ekappa function in stage- and lineage-specific rearrangement and somatic hypermutation. Immunity. 8(6):743-50.
16. Inlay M A, Gao H H, Odegard V H, Lin T, Schatz D G, et al. (2006) Roles of the Ig kappa light chain intronic and 3′ enhancers in Igk somatic hypermutation. J Immunol. 177(2):1146-51.
17. Wang C L, Harper R A, Wabl M (2004) Genome-wide somatic hypermutation. Proc Natl Acad Sci USA. 101(19):7352-6.
18. Yoshikawa K, Okazaki I M, Eto T, Kinoshita K, Muramatsu M, et al. (2002) AID enzyme-induced hypermutation in an actively transcribed gene in fibroblasts. Science. 296(5575):2033-6.
19. Arakawa H, Saribasak H, Buerstedde J M (2004) Activation-induced cytidine deaminase initiates immunoglobulin gene conversion and hypermutation by a common intermediate. PLoS Biol. 2: E179.
20. Arakawa H, Kudo H, Batrak V, Caldwell R B, Rieger M A, et al. (2008) Protein evolution by hypermutation and selection in the B cell line DT40. Nucleic Acids Res. 36(1): el.
21. Gopal A R, Fugmann S D (2008) AID-mediated diversification within the IgL locus of chicken DT40 cells is restricted to the transcribed IgL gene. Mol Immunol. 45(7):2062-8.
22. International Chicken Genome Sequencing Consortium (2004) Sequence and comparative analysis of the chicken genome provide unique perspectives on vertebrate evolution. Nature 432: 695-716.
23. Bulfone-Paus S, Reiners-Schramm L, Lauster R (1995) The chicken immunoglobulin lambda light chain gene is transcriptionally controlled by a modularly organized enhancer and an octamer-dependent silencer. Nucleic Acids Res. 23(11):1997-2005.
24. Kothapalli N, Norton D D, Fugmann S D (2008) Cutting Edge: A cis-Acting DNA Element Targets AID-Mediated Sequence Diversification to the Chicken Ig Light Chain Gene Locus. J Immunol. 180(4):2019-23.
25. Arakawa H, Lodygin D, Buerstedde J M (2001) Mutant loxP vectors for selectable marker recycle and conditional knock-outs. BMC Biotechnol 1:7.
26. Hamimes S, Arakawa H, Stasiak A Z, Kierzek A M, Hirano S, et al. (2004) RDM1, a novel RNA recognition motif (RRM)-containing protein involved in the cell response to cisplatin in vertebrates. J Biol Chem. 280(10):9225-35.
27. Buerstedde J M, Takeda S (1991) Increased ratio of targeted to random integration after transfection of chicken B cell lines. Cell 67: 179-188.
28. Schoetz U, Cervelli M, Wang Y D, Fiedler P, Buerstedde J M (2006) E2A expression stimulates Ig hypermutation. J Immunol. 177:395-400.
29. Naschberger E, Werner T, Vicente A B, Guenzi E, Töpolt K, Leubert R, Lubeseder-Martellato C, Nelson P J, Stürzl M. Nuclear factor-kappaB motif and interferon-alpha-stimulated response element co-operate in the activation of guanylate-binding protein-1 expression by inflammatory cytokines in endothelial cells. Biochem J. 2004 Apr. 15; 379(Pt 2):409-20.
30. Souto-Carneiro M M, Fritsch R, Sepúlveda N, Lagareiro M J, Morgado N, Longo N S, Lipsky P E. The NF-kappaB canonical pathway is involved in the control of the exonucleolytic processing of coding ends during V(D)J recombination. J Immunol. 2008 Jan. 15; 180(2):1040-9.
31. Wu C, Ohmori Y, Bandyopadhyay S, Sen G, Hamilton T. Interferon-stimulated response element and NF kappa B sites cooperate to regulate double-stranded RNA-induced transcription of the IP-10 gene. J Interferon Res. 1994 December; 14(6):357-63.
32. Jain A, Ma C A, Lopez-Granados E, Means G, Brady W, Orange J S, Liu S, Holland S, Derry J M. Specific NEMO mutations impair CD40-mediated c-Rel activation and B cell terminal differentiation. J Clin Invest. 2004 December; 114(11):1593-602.
33. Cartharius K, Frech K, Grote K, Klocke B, Haltmeier M, Klingenhoff A, Frisch M, Bayerlein M, Werner T. MatInspector and beyond: promoter analysis based on transcription factor binding sites. Bioinformatics. 2005 Jul. 1; 21(13):2933-42.
34. Quandt K, Frech K, Karas H, Wingender E, Werner T. Matlnd and MatInspector: new fast and versatile tools for detection of consensus matches in nucleotide sequence data. Nucleic Acids Res. 1995 Dec. 11; 23(23):4878-84.

1. Method for diversification of a target nucleic acid comprising introducing a genetic construct comprising a diversification activator (DIVAC) into a recipient cell to produce a recombinant recipient cell,
wherein said diversification activator is linked to the target nucleic acid within said recombinant recipient cell,
wherein said diversification activator is selected from the group consisting of a nucleic acid having the sequence SEQ ID NO:1, a fragment of said nucleic acid and a nucleic acid homologous to said nucleic acid and said fragment, and
wherein said diversification activator has the function of activating genetic diversification in transcription units linked thereto.

2. The method according to claim 1, wherein said diversification is somatic hypermutation.

3. The method according to claim 1, wherein the target nucleic acid is in addition linked to at least one nucleic acid capable of serving as a gene conversion donor for the target nucleic acid, and wherein said diversification is a combination of somatic hypermutation and gene conversion.

4. The method according to claim 1, wherein said diversification activator comprises an immunoglobulin enhancer sequence.

5. The method according to claim 1, wherein said diversification activator comprises a nucleic acid having the sequence between positions 2825-3625, 5114-6106, 5288-5485, 6107-7099, 6107-9784, 3098-7099, 9000-9784, or 9509-9802 of SEQ ID NO:1.

6. The method according to claims 1, wherein the length of said diversification activator is at least 100 nucleotides.

7. The method according to claim 1, wherein said diversification activator shares at least 50% sequence identity with SEQ ID NO:1 of a fragment thereof.

8. The method according to claim 1, wherein more than one copy of said diversification activator or more than one diversification activator is present in said recombinant recipient cell.

9. The method according to claim 1, wherein the target nucleic acid is an exogenous nucleic acid.

10. The method according to claim 1, wherein the target nucleic acid encodes an immunoglobulin chain, a selection marker, a DNA-binding protein, an enzyme, a receptor protein, or parts thereof.

11. The method according to claim 1, wherein the distance between the target nucleic acid and the diversification activator is no more than 150 kb.

12. The method according to claim 1, wherein said diversification of said target nucleic acid in the presence of said diversification activator is at least 5-fold higher than in the absence of said diversification activator.

13. The method according to claim 1, wherein the recipient cell is a eukaryotic cell expressing activation-induced deaminase (AID) or a functional equivalent thereof.

14. The method according to claim 1, wherein the recipient cell is a B lymphocyte or a cell line derived thereof, preferably the chicken B cell line DT40 or a derivative thereof.

15. Method for preparing a target nucleic acid having a desired activity, comprising the steps of:
(a) introducing into a recipient cell one or more nucleic acid construct(s) comprising a diversification activator and, optionally, the target nucleic acid to obtain a recombinant recipient cell,
(b) identifying a recombinant recipient cell, wherein said diversification activator is linked to said target nucleic acid,
(c) propagating and expanding the cell from (b) under conditions appropriate for the expression and diversification of said target nucleic acid, and
(d) selecting within the population of cells from (c) individual cells or cell populations containing a mutated target nucleic having a desired activity,
wherein said diversification activator is selected from the group consisting of a nucleic acid having the sequence SEQ ID NO:1, a fragment of said nucleic acid and a nucleic acid homologous to said nucleic acid and said fragment, and
wherein said diversification activator has the function of activating genetic diversification in transcription units linked thereto.

16. The method according to claim 15, wherein steps (c) and (d) are iteratively repeated.

17. The method according to claim 15, further including
(e) determining the mutant sequence of the target nucleic acid from the cells selected in (d).

18. The method according to claim 15, further including
(f) terminating said diversification.

19. A recombinant nucleic acid construct comprising a diversification activator, wherein said diversification activator is selected from the group consisting of a nucleic acid having the sequence SEQ ID NO:1, a fragment of said nucleic acid and a nucleic acid homologous to said nucleic acid and said fragment, and wherein said diversification activator has the function of activating genetic diversification in transcription units linked thereto.

20. A recombinant cell comprising a diversification activator as an exogenous nucleic acid, wherein said diversification activator is selected from the group consisting of a nucleic acid having the sequence SEQ ID NO:1, a fragment of said nucleic acid and a nucleic acid homologous to said nucleic acid and said fragment, and wherein said diversification activator has the function of activating genetic diversification in transcription units linked thereto.


SEQUENCE LISTING (119 sequences)

SEQ ID NO: 1 - DNA, 9784 bp, Gallus gallus
  misc_feature (1)..(9784): “W” fragment
ttccgccatggcctgggctcctctcctcctggcggtgctcgcccacacctcaggtactcgttgcgcccggtcggggactgtgggcacggggctctgtcccactgctgcgcgggcagggctgtgcgtgcggggccgtcactgattgccgttttctcccctctctcctctccctctccaggttccctggtgcaggcagcgctgactcagccggcctcggtgtcagcaaatccaggagaaaccgtcaagatcacctgctccgggggtggcagctatgctggaagttactattatggctggtaccagcagaagtctcctggcagtgcccctgtcactgtgatctatgacaacgacaagagaccctcggacatcccttcacgattttccggttccaaatccggctccacagccacattaaccatcactgggttccgagccgatgacgaggctgtctatttctgtgggagctacgaagacaacagtggtgctgcatttggggccgggacaaccctgaccgtcctaggtgagtcgctgacctcgtctcggtctttcttcccccatcgtgaaattgtgacattttgtcgatttttggtgatttgggggtttttcttggacttggcggcaggctggggtctgccaccggcgcagggccgggcactcagcgcggcagcctgggctgagtcttgtccccaccgagccggagggctccggtgtgcgccatggaggacttagggttattttgtcaatggaaagttcttaaaatttgaccagaaaatgtgcccgaggtctgtctctgccacacgatttcagaaattgtgtctaggtcgatgagaagacagtttttgtctttgtcaggaaattagttgtgagttgttagtccttccctcttagtcctaaggactaagacctttgtccccggtctggtctctcactggggactcttggctccagtgccatggggagcccaagtgtcactgacacggtgtccttgggggtgaaattcagtttttcagctgtccaggtaagtgctgtcaaaaaatggaggttattctgctgaaaaagctgagaggaatattttgtcatttttttcggaaatatatatatatatatatatataaatatataaattatttatatattttatatatatatatatataaaatatatgtatttctctctttctttttatatatatatataaatatatatatatttctccctctttctttatatatatttatagggagagaatctatatttttggccaatttggccaatttctctctctctctctttatatatatatatatatatatatgtaatttatatatatatatataaatttatatttatttatttatttatttatatagattatttttatatacatatatatatataaatttggccaatttagaagaaattggccaaaaaaacccaatctgaaccaaattacttagggcataagtccaaaaaaaaagtgcagttcaagaattcctcgctggaaagtttttaatgggggcagttcttccccattttctggtgtagatatgagtgaagatatagatatgtatgtagatatagatttggctatagtttgtttgatgaacagtctgaggaagagtaaatcttgccttcctcatggcaatttttaattctacttcttctgagacaagtctgaacatgccttgaggtagaaagagcccgaaaattgagtgctttttctttgaaattttaattaaaattaaaaaatagccttacagcaggaaagaaagggggtgatggcagctaatcggagttccaaagctgaaccacgttcacccaaacacgtggttgaaattttgtgtgtgtatatatataaatatatatatgtatacaaaagaaaatgcctatatataaaaaaattgttatgtatattatatagatttattatagatatgtacataagatataacacgtatacatatatatgtagtctgctgtatgctttgtataaatttttatagatatgatgtatattatatgcatgggttatatatctataatatattttatctgtagaaggaactacagtataaaatgcatatatgcgtatatatacacatatttatatatatgtgtatatatatgtatacgcacacgtataacatccatgaaaatagatatggatgggtggatgtgtttgttttacagaggtgcatgtgtgtctgtacatacacagccatacatacgcgtgtggccgctctgcctctctcttgcaggccagcccaaggtggcccccaccatcaccctcttcccaccgtcaaaggaggagctgaacgaagccaccaaggccaccctggtgtgcctgataaacgacttctaccccagcccagtgactgtggattgggtgatcgatggctccacccgctctggcgagaccacagcaccacagcggcagagcaacagccagtatatggccagcagctacctgtcactgtctgccagcgactggtcaagccacgagacctacacctgcagggtcacacacaacggcacctctatcacgaagaccctgaagaggtccgagtgctaatagtcccactggggatgcaatgtgaggacagtggttcctcaccctccctgtccctctgggccgctgctggtggcagcagccctcacttcccactcagatgtcccccaccgtgcccccatcacccacctctgcctgtcgactcctcttgccctcatctctccaggtgtcacattaataaacacgacactgaactagtgctgactctgcatccatgtctctgtgtccttttgcgtgctgtctgcatctcacacagtggggtcggccccagtatggggaagggctggggggcacatacacacatattggtaatgttgggggcgggggggggagggcgggggggtcaacagatcagcactggagacactggtgtataccctggcaacaccaacatctaaggcagggtgctttggggcaattttggggcagtttaaggtctgtgctggcactgagcacatggctgtggccgtgctgtcctcgtctcccacccactacggtctgtgcgccaggtccctagcagagatttgctttatgctgggaacagggggagttctgggtctgttcccttgcattcagacaccctggtgccccctgggtgggatgtcagtgtgaatactcctttgtgccctgtgcctgcagcagcctgaccctccacacaccacacgccttgtgtgcaccccacccctgtcactatccctctccccgctccccagggagattttgcagtggcccctgtagggcagcttttagcacagcccccagcagcaagcaagcagaaagcactgctgtgcacagcttgtcagctgtgtgtgtttgctgaggaggatctgtcttttgctgaggccatcagtcttgtcctgctcaacctccatcgatgctgcccacctcaacacatctacccatctattccatctacaccaacatctccattcatcccacccacccaaacatgtccatccatcacaacacctccatccaacccgcacactccagcacctccaatcattccatctacaccaccatgctgatctgctacagccactccaacgcaaccgtccattccatctacaccaatgtccatccatcccagccactccagcacctccagccatcccacccaccctatgtctccatccagccactggtggggtgcaggacatggggccagctctactgtcaggactggggtttttgcatggccccataccacttctgcagaagagacgcactgaaagtttggctgaccattttctccgcggtagagctgtggaaattctgtaatttagggtcttttatccagtttggagatgggctgggatctcccagctccatggcaggcattcatgacactgggtttagtatctgatgggtgggatgtggctgaacttcattttctttccccagtgacaaagtttttgcagttgaatatgaattcctgctttctgctctatgagttgtttttttcccaggacgtacacagggaatcagcagtcttcattctccctctgccatgtgtagactctctgccacacaggactgtgctgtcctcatgcccctgcgcccaaattgctgccctctgcccatgcctgccaagctgagcccccctgcaggctgccatgctggattgacatgagccctgagattggtacagaaatggtgattttggggttttctctgcactcaggaagctgaaggctcaatgctcagtgatggatttaccaaactgtgccctgaggcagctgctcatgctggataaagtcactggagcacagggaaccaggcgctgggcagggatttctcatgggccccacttggaaagctgcaggctgcaaacctggctctgccttcacgcctcaccctcatgaggacaacctcactaattattgattaaaagattttgctaaaccatctccagaagcaacaacccactgaggagcatgtgctgaattatacatcacagctccacggccctgccctcatagcagggctgcatggcacccacagtggcactcagcgggaccacagggctgagacagccgggtctggtggtggggacacagctgagcataggatgagccccccgggcagtgctgggctttgctaatgagcagaagtatggatagaaagcaaccccagggctccggtcccagctgcagctcttgctctgtcgtgtcctttggtgaaactttaaacagtcgcctttttttttctctttcttttctggcttgccattaatttcaaaccgagagagacctaatttagtaaatgagatgcttcaggaaggctttaattggctgcagatggaggcaggcagtgctatcgtggggcctggatcgcacagggggctgcatatcctcactagcagaatacactcaggctgggtccctcccacattcatgccccagaccagagggaatatgctctgttccccacacatctctcccaatcttgcagctgttgagccccaacatcccaccagcacacggggctcagcacgcctggcgacgtggcatcagcagagcaggccgcatggtacagctccatcagcacagctggggccacacaaagagctgggttactgtgggcagcaggctgaaacccgaaaacaagagctgggggctcagaatagccccgggagcaggcagggcctgggggagagggcaagcaccaggcccagggccacacagcccttccaggaaggcacagcgctgtcagggtgcagcacgctcagccccaccatgcagctgtgcggccggggcatccccaagctaaatttacttctcagtctccaatcagaaactgaagctgaggggcccacgccggccaaaaaaaggaaacgaaacagtctccagaaagcactgacgtgtgaagcagagcgagcgccgcgcaaaccggccgccatgtcacacacctcaggttggggctttgccagactgagctttgctgctgctcggggtgggtgcccacggcctgggcacatgggatggggtacacacgtacacacacttgcacacccacaccccaacacttcaggtgatgctggtgcagatgggtgccccccaggctgacccccccacgcatgggcctggccccacactgctccatccgtgtctctgtccccatgtgccacccctgcccgctcccaccacgcgtcaacccaaatcctgagttaatcccacgactcctgcctgcttccagcgtccatggcagactggagatgcccaaaatgcagagcaggtttccctgaatctgagagatgaaatggagttatgggtgttcccctgcggcggagccccagctgtaggaagctcagagccatcacacagcaattaaagaggaattaaattaaatcaataaatgttttaggcgggctcagctgccagcaccacctgaccgaaacagcccgcttgcaaagaggagagcatttgcatggctgtggcaaaacagcaaccgcctgttgtgcagctgggatggtgttatctggaaatgtacgcagcccaggaggggtaaacagctccaaactgagaccccgagcttgtccacaggttgtaaacaggctgacataaacacctttgtgccgtggaaaaatatttatcacctcaaatatagcaggttaataaaataaaactcccaacggagctacacacctgctttggaagggaagcagacacttgttttctgcttgatgttggctgtaggaagccatgtttcccgatgcaggagggccacaaagcactgacaacacaatgtgagctgagcttcgcccctgtttaagcccccacctcagggcttgtggcctcggagcaggcaggacgcaggggtggcaccgggctgggtgacatgggctggtcctggggtgtctcactgtgctctttggggaggggttggagccctggggcaatcacagcacacacaggaggtggggggatgcagccagcagctgccctgcactaagaaaaccccatccgtgggctttcagatggccttcccatctctctgcagcctctgcatgggctgagcacaaggtttaagtgtttctgccatgtttttgggcatgtttggaggggcagcatgggcccgggcatacgggtaccgccacgtgccgccagccccacagctgagcctgcactctcccagatgtgctgaccgcagccacgggggcaacagtttctcttgctaaaaattgtagccgggaagaaaacacgtggcaacttcggccaaacagcagctggaggacaggaatagccgtggccacggcacgctctgcttcctcggcacaaacattccagtatgtggcaccacgagcgccgctgcccggcacagcagcaagcagagccaggagcaggaaatgctgatttgggccccattttggccatggctgagagaagaggcttccagggagctggtcagcttggtccccaagctgtggcttggggaaatgatggggaggggattgccactgcccaccctgcagagcaggctctggtcccatctcactgcagggcaccagggcgtttgcactgcagcaattcacagaaacattgaaatggctcctgccttgttcaacatcttcatcagtgacctggatgaggggacagcatccaccatcagcgggttcactgatcatatgaagtcgggaggagtggctgacgcaccacaaggctgtgctgccattcaacaggacgtggacagactggagagctggacagggaggaacccaatgaggttcaacaatggcaagtgtaggatctacacctgggaaggaataacagcatgcatcagttcaggttaggggctgagctgctgcagatgagctctgagagaaggacctgagcatcctgctggacagcaggctggctgtgagccaccggtgtgccctggtggccaagaaggccagtggtatcctggggagcaccgcaatgagagtgggcagcagggcgagggaggtgaggctgcatttggagcaccgtgcccagttctgggctcctcagttcaaggcagacagggaactgctggagagagcccagcagaggggctgcaatgatgatgagggtcctggagcatcgcctgtatgaggaaaggctgagggacctgggattgttcagcttggagaagagaagactacagggcaggagccaagtggatagggccgggctcttttcagcagtgcccagtgacaggacaaggggcagcaggcacaaagtggaacataagaagttccatctgaacatgaggaaaaactgcctcgctttgagggtgaccgagcactggaagaagctgcccagagaggtggtggagtctcctctggagatattcagagcctggcaggacactttttgctgagtaacctactgtagggaacctgacgcagcagaggggtcggactggaggatctccggaggtctctttcaacccctacagttccatgaaatacctcaaacactgccaagcgcagtgctaaggcaagggtaacatttgtaaactgaaacagggtgggtttaagttagatgtaaagaagaaactcttcactcagagggtggcgaggccctggcacaggctgcccatggaggctgcgggtgccccatccctggcagtgcccaaggcaagagcccagcagtgaccacagccccacaaggacgagcgtggccctcgtatctcagctcaccctgccccagctcaactcccacctccggcacagcgcgggcacacagccgggccctgtgcttatggagcccttggggcaggtcagcactcacaccctccaaacacagccgtggctcccaaccggaggcagctggatctcggcagccataaccaagcagggccatgcgggggtgacaccggggtcccccaccccctgtggggcagcgtatgggctgggcccctgctccagtctgcagcgtgtgcatgggaaccattgccagacaccgtctggaccacccgcagccctaagctgcctcacagcagggattgctccgtcacaccgtgaccccgtgcccttattccatcacttatggggctgggagtgcctggaccttgggcacattaacgaggatttcccgctctgccctcgctttgctccgagccgtggggctgtgtagtgcagacacagctgcagcctaaaattagtacctgggaaaggcccccatgctgcaccgcgcagggctgagatgtgccacgtccccatggccggagctggggaaggcaacgtggccctgtgcgtgtgcacgctgagcacaaggacacgtgctgggccaggatttgtctccccggggctcacgctatgtgtcacccggtgctgcgccatcccctcccgcagcccccagctcccccacggccgcacgccgcctgcatccctgcaacggcaccgcacagagacacggagccaggggccgcacacggggccaggagctcacctttattgcagccctgacagccccacggcccagcccgcaccggggctgccacatcctcacccggccgacggcccccagctgctccttaccatttcttccccccatcaccccataaaccagaagccgcctcaccgctacgcggagcgggcagcagggaacccgggccctaagggggagacgagagggggccgagcaggggcaggaggagcagcagggcgagggggcagcgggggcacccacagctggccgtggcatcccgggaggagaagaccttgcggctgcggagcggttgtggcggacggaaattgttggtcatcttcaggggcgcagcgcccgaggccgggaagtgcacggtgctgacaaacgcctgcagctgcggggagagcaccgcgggcgccgcagccgtgaggcgtagggcgaagcggggcacacgcgtggctgctgctgtctttccccctaccggcagcacacggctctgcacacaccgcgcttcgtgccgcctcgcagccgacgctgcaggaagcccagccgagcgcttacagagcggccgggaaatgcatctgctgaggtgcccgggcaatgcagaacttcatccatccccacatccattcaccagtcccctcccaaacccccaagcccatccggcgacccacccaccctcctcttggtgcccctctcaagctctccatccccacattcctacagatgtcccctttactttgcctgcaaggtgcaagaaaacgcacaggaccgggggtgctcacagcacggctttggccagacgggcccttccatccca

SEQ ID NO: 2 - DNA, 34 bp, Gallus gallus
ggggtcgacatatgatacacagacctgacatctc

SEQ ID NO: 3 - DNA, 40 bp, Gallus gallus
gggggatccactagtatcacataatcaccaaggttggaaa

SEQ ID NO: 4 - DNA, 34 bp, Gallus gallus
gggggatccttccgccatggcctgggctcctctc

SEQ ID NO: 5 - DNA, 34 bp, Gallus gallus
ggggctagccctctcagctttttcagcagaataa

SEQ ID NO: 6 - DNA, 34 bp, Gallus gallus
gggactagtaaaatgatgcataaccttttgcaca

SEQ ID NO: 7 - DNA, 30 bp, Gallus gallus
cccaccgactctagaggatcataatcagcc

SEQ ID NO: 8 - DNA, 39 bp, Gallus gallus
gggctcgagggtactgcgttttccacaaaattctcacag

SEQ ID NO: 9 - DNA, 39 bp, Gallus gallus
gggagatctctgcactctggcaccgttaagcaccatcac

SEQ ID NO: 10 - DNA, 38 bp, Gallus gallus
gggtgatcaagatctgctagcactagtggatccgtcga

SEQ ID NO: 11 - DNA, 39 bp, Gallus gallus
ggggaaaagcggccgccactggaaggagctgaaggccac

SEQ ID NO: 12 - DNA, 27 bp, Gallus gallus
agcttggaatttaacctctcctgtaaa

SEQ ID NO: 13 - DNA, 30 bp, Gallus gallus; identical to SEQ ID NO: 7

SEQ ID NO: 14 - DNA, 34 bp, Gallus gallus
gggatcgatttccctggtgtgggtttttttgggt

SEQ ID NO: 15 - DNA, 34 bp, Gallus gallus
gggatcgatttgagaaagctcacgctccttccaa

SEQ ID NO: 16 - DNA, 34 bp, Gallus gallus
gggactagtcatctcagcggtgcttatgaatgac

SEQ ID NO: 17 - DNA, 34 bp, Gallus gallus
gggactagtaccccaaaaagcagccgagcatttc

SEQ ID NO: 18 - DNA, 30 bp, Gallus gallus
gctgctcaccttccttaccctgctgctcct

SEQ ID NO: 19 - DNA, 30 bp, Gallus gallus; identical to SEQ ID NO: 7

SEQ ID NO: 20 - DNA, 34 bp, Gallus gallus
tttatcgatggagaagtgagcggcagagggaaat

SEQ ID NO: 21 - DNA, 34 bp, Gallus gallus
tttatcgattcagcagggcaatgggcacacactg

SEQ ID NO: 22 - DNA, 34 bp, Gallus gallus
tttactagtgcgcagcagagcgcaccaaccacag

SEQ ID NO: 23 - DNA, 34 bp, Gallus gallus
gggactagttcgacagcgagtgcaaaatcatctt

SEQ ID NO: 24 - DNA, 28 bp, Gallus gallus
ggaccgggggtgctcacagcacggcttt

SEQ ID NO: 25 - DNA, 30 bp, Gallus gallus; identical to SEQ ID NO: 7

SEQ ID NO: 26 - DNA, 33 bp, Gallus gallus
gggactagttgcccttttgcttgcagccagcct

SEQ ID NO: 27 - DNA, 34 bp, Gallus gallus
gggctcgagaatctcaacagctgtgaagtttcga

SEQ ID NO: 28 - DNA, 28 bp, Gallus gallus
ggggaaacagtgagcatggggattccct

SEQ ID NO: 29 - DNA, 30 bp, Gallus gallus; identical to SEQ ID NO: 7

SEQ ID NO: 30 - DNA, 33 bp, Gallus gallus
gggtctagaagtagtccaaccaacctatgcagt

SEQ ID NO: 31 - DNA, 33 bp, Gallus gallus
gggctcgagtctgaagcctgaaatcacacagca

SEQ ID NO: 32 - DNA, 25 bp, Gallus gallus
ctttctatccgtgcttagtctggtt

SEQ ID NO: 33 - DNA, 30 bp, Gallus gallus; identical to SEQ ID NO: 7

SEQ ID NO: 34 - DNA, 33 bp, Gallus gallus
gggctcgagtagtctgtgcatgaaaatgtgctg

SEQ ID NO: 35 - DNA, 34 bp, Gallus gallus
gggggatccagcaattaaccaaatcctctgacag

SEQ ID NO: 36 - DNA, 34 bp, Gallus gallus
gggggatcctcgctcctttattctctccagtggc

SEQ ID NO: 37 - DNA, 34 bp, Gallus gallus
gggtctagacagccagcctatgcactgcctccac

SEQ ID NO: 38 - DNA, 25 bp, Gallus gallus
gtaacactgataagagagagatcag

SEQ ID NO: 39 - DNA, 30 bp, Gallus gallus; identical to SEQ ID NO: 7

SEQ ID NO: 40 - DNA, 39 bp, Gallus gallus
gggctcgaggtcatctgagagagaacccagctgacatgg

SEQ ID NO: 41 - DNA, 38 bp, Gallus gallus
gggggatccgcttcacaacttaacagaggtaggtttca

SEQ ID NO: 42 - DNA, 38 bp, Gallus gallus
gggggatccgtgagagtactgaactgagtcctggacag

SEQ ID NO: 43 - DNA, 39 bp, Gallus gallus
gggactagtcagtcaacatcaggcaggaagatctggttt

SEQ ID NO: 44 - DNA, 30 bp, Gallus gallus
gagcctgtgaggcaacttctgtgcaaccca

SEQ ID NO: 45 - DNA, 30 bp, Gallus gallus; identical to SEQ ID NO: 7

SEQ ID NO: 46 - DNA, 34 bp, Gallus gallus
gaactcgagtgcctgcggggagcgcgcagacatt

SEQ ID NO: 47 - DNA, 41 bp, Gallus gallus
gggggatccttagtagaacttgatgatggcatagcagccag

SEQ ID NO: 48 - DNA, 34 bp, Gallus gallus
gggggatcctctggagtttggcacagccacaaga

SEQ ID NO: 49 - DNA, 39 bp, Gallus gallus
ggggctagccatatgaggtagatgtcattgcacagcttt

SEQ ID NO: 50 - DNA, 39 bp, Gallus gallus
gaaagatctaggggggcccgggcatggcggaggtgttgg

SEQ ID NO: 51 - DNA, 30 bp, Gallus gallus; identical to SEQ ID NO: 7

SEQ ID NO: 52 - DNA, 39 bp, Gallus gallus
ggggaattcatatcctccgctgtctcattgacatacatt

SEQ ID NO: 53 - DNA, 39 bp, Gallus gallus
gggggatcctttgctgtatattaatacttgatggaaaca

SEQ ID NO: 54 - DNA, 39 bp, Gallus gallus
gggggatcccaagcagttaataagcttccacgtcagatg

SEQ ID NO: 55 - DNA, 39 bp, Gallus gallus
gggtctagagtcgcagtttccgctgatacgtggcatcac

SEQ ID NO: 56 - DNA, 30 bp, Gallus gallus
cttgccaagggtacagctagcatccctctt

SEQ ID NO: 57 - DNA, 30 bp, Gallus gallus; identical to SEQ ID NO: 7

SEQ ID NO: 58 - DNA, 40 bp, Gallus gallus
gggctcgaggatgactttcaatatgcagatcatgactacg

SEQ ID NO: 59 - DNA, 35 bp, Gallus gallus
gggggatccctctgatcttcttatttagtccaagg

SEQ ID NO: 60 - DNA, 45 bp, Gallus gallus
ggggtcgacggatccaattaagaagaaactgaatgcgtgttcttc

SEQ ID NO: 61 - DNA, 35 bp, Gallus gallus
ggggtcgacgctagctcacagtatcagagcccttg

SEQ ID NO: 62 - DNA, 30 bp, Gallus gallus
gagaccgcgcacgagcgaagaagatgatga

SEQ ID NO: 63 - DNA, 30 bp, Gallus gallus; identical to SEQ ID NO: 7

SEQ ID NO: 64 - DNA, 34 bp, Gallus gallus
gggatcgattccttggcatacgcccatggtttgg

SEQ ID NO: 65 - DNA, 37 bp, Gallus gallus
gggatcgattcaggccatcttgagtctgcccaggacg

SEQ ID NO: 66 - DNA, 34 bp, Gallus gallus
tttactagtggcagcctgagctggtgggggcaag

SEQ ID NO: 67 - DNA, 34 bp, Gallus gallus
gggactagtggaattctgaagaatcattctggtg

SEQ ID NO: 68 - DNA, 30 bp, Gallus gallus
gagctgtctctgaaagaagcggtgagaaaa

SEQ ID NO: 69 - DNA, 30 bp, Gallus gallus; identical to SEQ ID NO: 7

SEQ ID NO: 70 - DNA, 39 bp, Gallus gallus; identical to SEQ ID NO: 8

SEQ ID NO: 71 - DNA, 39 bp, Gallus gallus; identical to SEQ ID NO: 9

SEQ ID NO: 72 - DNA, 38 bp, Gallus gallus; identical to SEQ ID NO: 10

SEQ ID NO: 73 - DNA, 39 bp, Gallus gallus; identical to SEQ ID NO: 11

SEQ ID NO: 74 - DNA, 27 bp, Gallus gallus
agcttggaatttaacctctcctgtaaa

SEQ ID NO: 75 - DNA, 30 bp, Gallus gallus; identical to SEQ ID NO: 7

SEQ ID NO: 76 - DNA, 35 bp, Gallus gallus
gaagctagcttccgccatggcctgggctcctctcc

SEQ ID NO: 77 - DNA, 49 bp, Gallus gallus
gaaactagtattttttgacagcacttacctggacagctgaaaaactgaa

SEQ ID NO: 78 - DNA, 34 bp, Gallus gallus
ggggctagcggtggatgtgtttgttttacagagg

SEQ ID NO: 79 - DNA, 34 bp, Gallus gallus
gaagctagcgcaaatctctgctagggacctggcg

SEQ ID NO: 80 - DNA, 34 bp, Gallus gallus; identical to SEQ ID NO: 78

SEQ ID NO: 81 - DNA, 34 bp, Gallus gallus
gaagctagcgtgtggcagagagtctacacatggc

SEQ ID NO: 82 - DNA, 34 bp, Gallus gallus; identical to SEQ ID NO: 78

SEQ ID NO: 83 - DNA, 34 bp, Gallus gallus
gaagctagcatggagctgtaccatgcggcctgct

SEQ ID NO: 84 - DNA, 34 bp, Gallus gallus; identical to SEQ ID NO: 78

SEQ ID NO: 85 - DNA, 34 bp, Gallus gallus
gaagctagcaagctcagggtctcagtttggagct

SEQ ID NO: 86 - DNA, 34 bp, Gallus gallus; identical to SEQ ID NO: 78

SEQ ID NO: 87 - DNA, 34 bp, Gallus gallus
gaagctagcattgctgcagtgcaaacgccctggt

SEQ ID NO: 88 - DNA, 34 bp, Gallus gallus; identical to SEQ ID NO: 78

SEQ ID NO: 89 - DNA, 34 bp, Gallus gallus
gggactagttgttcagatggaacttcttatgttc

SEQ ID NO: 90 - DNA, 34 bp, Gallus gallus; identical to SEQ ID NO: 78

SEQ ID NO: 91 - DNA, 34 bp, Gallus gallus
gaagctagcatgggatggaagggcccgtctggcc

SEQ ID NO: 92 - DNA, 34 bp, Gallus gallus
gaagctagctttatgctgggaacagggggagttc

SEQ ID NO: 93 - DNA, 34 bp, Gallus gallus; identical to SEQ ID NO: 91

SEQ ID NO: 94 - DNA, 34 bp, Gallus gallus
gaagctagcaggactgtgctgtcctcatgcccct

SEQ ID NO: 95 - DNA, 34 bp, Gallus gallus; identical to SEQ ID NO: 91

SEQ ID NO: 96 - DNA, 34 bp, Gallus gallus
gaagctagccacgacagctggggccacacaaaga

SEQ ID NO: 97 - DNA, 34 bp, Gallus gallus; identical to SEQ ID NO: 91

SEQ ID NO: 98 - DNA, 34 bp, Gallus gallus
gaagctagcgtcacaggttgtaacaggctgacat

SEQ ID NO: 99 - DNA, 34 bp, Gallus gallus; identical to SEQ ID NO: 91

SEQ ID NO: 100 - DNA, 34 bp, Gallus gallus
ggggctagctcacagaaacattgaaatggctcct

SEQ ID NO: 101 - DNA, 34 bp, Gallus gallus; identical to SEQ ID NO: 91

SEQ ID NO: 102 - DNA, 34 bp, Gallus gallus; identical to SEQ ID NO: 92

SEQ ID NO: 103 - DNA, 34 bp, Gallus gallus
gaaactagtattgctgcagtgcaaacgccctggt

SEQ ID NO: 104 - DNA, 40 bp, Gallus gallus
aaatgatcacccctctccctcccccccccctaacgttact

SEQ ID NO: 105 - DNA, 40 bp, Gallus gallus
gggtgatcaggatccgatccagacatgataagatacattg

SEQ ID NO: 106 - DNA, 41 bp, Gallus gallus
gggggatccagatctgtgaccggtgcaagtgatagaaaact

SEQ ID NO: 107 - DNA, 51 bp, Gallus gallus
tacaaaaacctcctgccactgcaaggagcgagctgatggtttttactgtct

SEQ ID NO: 108 - DNA, 34 bp, Gallus gallus
gaggctagcagctgtgcggccggggcatccccaa

SEQ ID NO: 109 - DNA, 43 bp, Gallus gallus
gagactagtggcgcgccagctcagtctggcaaagccccaacct

SEQ ID NO: 110 - DNA, 44 bp, Meleagris gallopavo
gctagcactagtcagcatgctcagccccaccgtgcagctgtgtg

SEQ ID NO: 111 - DNA, 42 bp, Meleagris gallopavo
actagtgctagcaagctcaatctggcaaagctccaacctgag

SEQ ID NO: 112 - DNA, 42 bp, Cairina moschata
gctagcactagtaggtgtggggtcggggcgtccccgagctaa

SEQ ID NO: 113 - DNA, 46 bp, Cairina moschata
actagtgctagccacggcgctcgcttggcgcggcgctcgctctgct

SEQ ID NO: 114 - DNA, 218 bp, Gallus gallus
  misc_feature (1)..(218): 2.2-2.4 fragment
gctagcagctgtgcggccggggcatccccaagctaaatttacttctcagtctccaatcagaaactgaagctgaggggcccacgccggccaaaaaaaggaaacgaaacagtctccagaaagcactgacgtgtgaagcagagcgagcgccgcgcaaaccggccgccatgtcacacacctcaggttggggctttgccagactgagctggcgcgccactagt

SEQ ID NO: 115 - DNA, 240 bp, Meleagris gallopavo
  misc_feature (1)..(240): 2.2-2.4 fragment homologue in Turkey
actagtcagcatgctcagccccaccgtgcagctgtgtggccggggcatccccgagctaaatttactcctcagtctccaatcagaaactgaagctggggggcccacgctggccaaaaaaaggaaatgaaacagtctccagaaagcactgacgtgtaaagcagagcgagtgccgcgcaaacttgccgccatgctacatgcctcaggttggagctttgccagattgagcttgctagcactagt

SEQ ID NO: 116 - DNA, 178 bp, Cairina moschata
  misc_feature (1)..(178): 2.2-2.4 fragment homologue in Duck
actagtaggtgtggggtcggggcgtccccgagctaaatttactccccagtctccaatcaaaacttgcgctggggggcccgagctggcagaaaaaaggaactgaaacggtctccagcaagcgctgacgtgtgaagcagagcgagcgccgcgccaagcgagcgccgtggctagcactagt

SEQ ID NO: 117 - DNA, 8053 bp, Meleagris gallopavo
  misc_feature (1)..(8053): “W” fragment homologue in Turkey
tcccactggggatgcgatgtgaggacggtggttcctcaccctccctgtccctctgggccactgctggtggcagcagcccccacttcccactcagatgtcccccaccgtgcccccaccacccacctctgcctgttgcctcctcttgcctccatccctccagatgtcacattaataaacatgacactgaactagtgctgactctgcatccatgtctctgtgtccctttgcatgctgtctgcgtctcacagatggggtcggccccagtatggggaagggttggggggcacatacacacgtactggtaatggtgggggggtccatggctcagcactggagacactgctgtatacccaggcaccaacaacatctaaggcaggatgctttggggcaattttggggcagtctaaagtctgtgctggcagtgagcatatggctgtggctgtgctgtcctcatctcccacccactgcagtctgtgccaggtcctttgcagagatttgctttatgctgggcacatgggtagttctagctctgttcccttgcattcagacaccctggtgtcccctgggtgggatgtcagtgtgaatactcctttgtgtcctgtgcctgcagcagcctgactctccacaccccagccctgtccctatccccattcccgctccccagggagattttgcagtggtccctgtagggcagctctgagcacagcctgcaagccagcaagcagaaagcactgatgtgcacagcttgtcagctgtgtgtcctccttgctgaggaggatccttactgagtccatcagtcttgtcccactcaatctccatcgatgctgccgacctcgacacatctacccatctattcaatctacaccaatatctccattcatcccacccactcaaacacgtccattcatcacatctatcacaacacctctatccagcctgcacactccagcacctccaatccttccatctacactaccatgctgatctgcctcagctactccaacacaaacatccattctgtctacaccaatgtccatccatcccagccactccagcacctccagccatcccacccaccctatgtctccatccagccactggtggagtgcaggacatggggccagctctactgtcaggactgaggtttttacatggccccatatgcactgaaagcttggctggccattttctctgcagtagagtttgacaattctgggggacttttatccaggttgcagaagggctgggatctcccaggtccatgggcaggcatttgtgacactgggtttagtgtctgatgggtgggatgtggctgaacttcattttctttccccagtgactaagtttttgcagttgaatgtgaattcctgctttccgctctattagttgtttttttttcaggatttacaagggaaatcagcagtcttcattctcccctcgccatgtttagactctctgtgctgtcctcatgcccctgtgcccaaatcactgctctctgcccatgcctgccaagctaagccctgcaggctgtcatgctggattgacatgagccctgagattggtacagaaagtatttctgtaccaatttctgtgactttggggctttttccacactcagggagctgagagcttagttcttagtgatggatttaccaaattgtgccctgaggcagctgctcatgctggataaaatcaccaaagcacagggaaccaggcgctgggcagggatttctcatgggctccactttgaaagctgcaggctgcaagccaggctctgcctttgcacctcgcactcgtaaggattacctcactaattattgattaaaagattttgctaaaccatttccagaagcaacaacacactgaggagcatgtgctgaattatacatcacagcactatggccccggccctctcagcagggctgcatggcacccacagtggcactcaatgggaccacagagctgggacagccaggtctggcagtggggacacagttgagcccaggatgagcccccccgacagtgctgggctttgctaatgagcagaaacacggatagaaagcaaccctggggctctgcacccagctgcagctcttgctctgtcgtgtcctttggtgaaattttaaacagtcacctcttttttttttcttttatagcttgccattaatttcaaaccaagagagacctaatttagtaaatgagatgcttcaggaaggctttaattagctgcagatggaggcaggcagtgctgtcgtggggcctggatcacatagggggctgcgcaccctcactatcagaatacacccaggttgggtccattccacatttgtgccccagtccagaggggatgtgcttttgttccccacacatcactcccaatcttgcagccgttgagccccaacatctcaccagcacatgggactcagctggcctggcgacgtggcagcaacagagcaggccgcgtggtacagctccatcagcacagctggggccacacaaagagctgggttactgtgggcagcaggctgaaacccgaaagcaagagcttggggctcagaatagccctgggagcaggcagggtctgggggtgaaggcaagcaccagtcccagggccacccagcccttccaggaaggcacagctctgtcaaggtgcagcatgctcagccccaccgtgcagctgtgtggccggggcatccccgagctaaatttactcctcagtctccaatcagaaactgaagctggggggcccacgctggccaaaaaaaggaaatgaaacagtctccagaaagcactgacgtgtaaagcagagcgagtgccgcgcaaacttgccgccatgctacatgcctcaggttggagctttgccagattgagcttgctactgcttgggtgggtgcccacggcctgggcacacggcacaaggtacacacgtacacacacttgcacacccacacaggccaaaccatcaggtgatgggtgccctccaggctgggcccacactgctcaatctgtgtctttgcccctatgtgcctcccctgcctgctcccaccacacgtcaccccaaatcctgagttaatcctgcgactcctgcctgcttccagcatctgtggcagactggagatgcccaaaatacagagcaggctttcctgaatctgggagatgaaacggagttataggtgttcccctgcagcagagcctcagctgtaggaagtacagagccatcacactgcaattaaagaggaattaaattaaatcaataaatgttttaggcgggctcagctgccagcaccacctgacccgaaacagcctgcttgcaaagaggagagcatttgcatggctgtggcaaaacagcaaccgcctgttgcgcagctgggatggtgttatctggaaatgtacacagcctgtgaggggtaaacagctccaaacaaagaccccgagcttgtcaacaggttgtaaacaggctgaaataaacacctttgtgctgtggaaaaatatttatcatctcaaatttagcaggtttataaaataaaactcccaacagagctacacacctgctttggaagggaagcaaacacttgttttctgcttgatgttgactgttagaagccatgttttcctgatgcaggtgggccacaaagcaatgaaaacacaatgtgagctgaacttcggccctgtttaagtccccacctcagggcttgtggcctgggagcagacagtgcacagggtctgggcacggggtggcacagggctgggtgacatgagctggccctggggtgtctcactgaactctttggggaggggtttgagccctggggcaatcacagcatgcacagaggaggcgggaggatgcagccagcagctgctctgcgctgagcaaaccccatctgtgggctttcagatggccttcccgtccctctgcagcctctgcatgggctgagcacatggcttaagcatttctgccatgtttttggcgtgtttgaaggggcagtgtggacacgtgggtaccgccacgtgcagccagcgccacagctgagactgcaatctcccagatgtgctgaccgcagccacagggcccgggggcagcagtttctcttgctaaaaattgtggccgggccgaaaacatgaggccacttcggccaaacagcagctggaggacaggaatagctgtggccatggcatgctctgcttcctcggcacaaacattccagcacgtggcaccatgagtgccactgcccggcacagcaggaagcagagccaggagcaggaaatgctgatttgggccccattttggtaatggctcagagaagaagcttccaggaagctcaccagtgttggtatttggggcttggagaaatggtggggagggaatagccactgcccccgttccagagcaggtactggtcccatctcgctgcagggcaccagggcacttgtactgcagcaatacacagaaacacctgaaatggctcctgtcatgttcagcatggctccggtcttgttcaatatcttcattagtgacctaaatgagtggcagcagtgggtgcatgggaactattgccagacattgtctggatcacctgcacacctaagctgcctcacaccaggggattgctcagtcccaccgtgaccctgtgcccttattccataacttagggagctggagtgcctggacttggggcagacactgctgccattcaacaggatgtggacagactggagagctggacagagaggaacccagtaaggttcaacaagagcaaggatcctacacctgggaaggaataacagcatgcatcagttcaggttaggggctgagctgctgcaaatgagctctaagagaaggacctgggtgtcctgctggacagcaggctggctgtgagccagtggtgtgcccttgtagtcaagaaggccagtggtatcctggggggcactgcaatgagagtgggcagcagtttgagagaggtgaggccacgtttggagcaccgtgcccagttctgggctcctcagttcaagacagaccagagaactgctggagagagcccagcagagggctgcaaagatgatgagggtcctggagcatcccctgtatgaggaaaggctgagggacctgggactgttcagcctggagaagagaagaccagagggcaggagccaagtggacagggccaggctcttttcagcagtgcccagtgatgggacaagggcagtaggcacaaactggaacttaagaagttctgtctgaacatgaggaaaaactgcttcgctttgagggtgaccgagcattggagaagctgcccagagaggtggtggagtgtcctctggagatatttagagccaggacgctttgctgtgtaacctactgcagggaacctgattcagcaggggggttggactggaggatctctggatgtctcttttaaccccaacaattccgtgactttgtgaaatacctcaaatactacaaagcgtagctctaagacaagagtaacatttttaaactgaaagatggtgggttttagttagatgtaaagaagaaattcttcactcagagggtggtgaggccctggcacaggctgcccaaagaagctgtaggtgccccatccctggcagtgcccaaggcaagaagcccagcagtgaccacagctccacaaggatgagcatggccccttgtatctcagctcaccctgccccagctgaactcccacctccagcacagcatgggccctgtgcttatggagcccttggggcaggtcagcacccacatcccccgaacacggccgtggctcccaaccggaggcagctggagctcggcagccacaaccaagcagggccacgcggggatgacaccagggtctcccagccccctgtggggcagcacgtcgagggctgggcccctgctccagtctgcagtgggtgcatgggaactattgccagacattgtctggatcacctgcacacctaagctgcctcacaccaggggattgctcagtcccaccgtgaccctgtgcccttattccataacttagggagctggagtgcctggatttggggcagacacagctgcagcctaaaattagctcctgagaaagggcccccaggctgcaccgagcaggactgagatgtgccacgtccccaaggccagagctggggaaggcaacgtggccatgtgtgtgtacgctgagcacaaggacacgtgctgggccaggatttgtctccttgagattcacgctatgtgtcacccggtgccgtcccatcccctcccgcagcacccagctccccacggccgcacagatgtgtgtgtgtgttcctgcaacggcaccgcacagagacacggagccaggggccgcacacggggccagcagctcgcctttattgcagccctgacaaccccatgatcgagcccacaccagggctgccacatcttcacccggctgacggccccagctgctttttaccattttttccccatcacccataaaccagaagtttcctcaccactatgcaaagaacagggaacagggaacccgggccctaaggggaagacgagaaggggccgagcaggggcagcaggagcagcggggcgaagggacagcggggacacccacagctggccgtggcatcccgggaggagaagaccttgcggctgcggagcggttgtggcggacggaagttgttggtcatcttcaggggcgcagcgcccgaggccgggaagtgtacggtgctgacaaacgcctgcagctgcggggagaggtgcgggtggaagcagcaccgcgggcactgtagccgtgaggcgcggggtgaagctaggcacacacacagatgctgccgagcagagcgcagcgcaggagccccgtctttccccctaacggccagcacacggctctgcacacactgcgctttgtgccgcctcgcagctgatggtgcaggaagcccagcagagcatttacagagtggccaggaaatgcatctgctgagatgcccgggaaatgcaggaggacttcatccatccccacatccattcaccaatcccttccccaaccccccgcccatccagcaacccacccactttcctcttggtgcccctctcaaactctccatccccgcatttctgcagatgtcccctttactttgcctgcaaggcgcaagaaaatgcacagggactggggatgctcacagcacggctttggctagataggcccttccatcccatggcaggtaatcccattacctgctccctgctgatgcccacgggctcctcaaacacagtccagatgacagcctcgctgcagtcaggagtggtcagagagccctggtagcggtagtaccgggagagctgcgcgatgtgcggcagcagcgtgccaaggcggaaggtggaggccaggtccactgcctgccctgcacagcaggacgtggacactcagctccaccacccccagcatgccccagggtctctcccctcccaatcgtgtcccagcgcagagccccagggcacctcaccagcgtgggagatgttcctcagtcccccaatgatagtgttgtagttggagtttgcggcttctgagacctgcaaagagtgagtgggccctgcagttgtgctcagctaagaccaatgcatggcagcacccatcccgtgcacttgcagccaacctggaagaagcagcccagcacagccagcccacttgggtgccccttggcctcccccaaggtgcggtacttgacgttgatgtggacgatgtgcagctggagaaagtacggagggctcagcatggacacctggccttgccagcagccaggcactaggatgggaaatcaaagggctgtggtactaaggggctttttgcctacctccatggggagctgttgcccatccacggtgtgctcagagccattcctagatgggctgccccagtggaagtgcagctgcaatgcacggtaccggcccgggaggcccccaccactgatggcaatgtgctcggcacccggctcactctccaggctcagcatcactgtaaatgataggagagggacagggctgggggccatggggcagcacctc

SEQ ID NO: 118 - DNA, 9053 bp, Cairina moschata
  misc_feature (1)..(9053): “W” fragment homologue in Duck
gcccgacggggcacagccatgaagacagtggctcctcactcttcccatccctcttggccactgctggtgacagcagccccccctcctcacccggatgtccctcttcatgcccccgccacccccccatgcctgtcccctgctcctgtccccctccgccgggtgtcacacgaataaacaccaacactgaactagcgctggctctgcatccgtgtctctgtgtccttgtgcgcgccaaccgccccgtgcggagcggggtcagccctggtgtgggggagggttgggggggaagggcacacactgggagtgctgggagtgcttgggggttcctggctgagcactgggaacactgatgcacccccctggcacctcctacaccaaaggcagggcactctgaggcagtttggggcagtctaagcaccgtgctggcactgagcgcttggccatggccttgtccctcactcactggcgtctgggtgccgggtccctcacagggacttgctgcccttgctgcgtgtgctggggacatgggcagtgctggctccgtccctgtgtgctcagtcaccccggtgtcccgtgggtggagatgccggagtggccgctcacacgttacgctgtccccgtgcctcatgcctgcagcagcctggtccccacaccatgtgcccctgtgtgtgtcccatccccataacggcctcagggaggttttcctgtggcccccacagggcagctctgggcagacatccttgcagccagcaagcagaaagatctgtgtggctgtcggccctgcgtgctcccaccaagtctgtcttgattgcctcaatgtctccatccatccagcccaccccagcacatctgcccatccaccccatttacaccaccatctccaccccatttacactccatctccagtccatgcaaacacctccatctatcacgtccaccacaacacctccacacattcatccgttcatccctctagcccatctacaccaacacctccctctactcacccacccacaccaacacctccatctgtcccaccaccctgccctccgcccaccccatgtctccatccagctggtggtgggtgcaggagcaggggccggccacgctctcaggacagagatttttgtatggccttaaaccgatgctggaggagggacacagacacctgcacttggtgcagcactgaagccatgtctggctgttttcttcactgtggagttgtgacatttcagtgatgtgggggaacttttctccagtttgggaaggggctggggtctcccagccccattgatgtgcactcaatggcactgaggacagtgttcgatggatgggattgggctgaatttcattttcttcctccagcaattgagttttgtcagtggaaggtgaaatcctgctttgtgccccgtgcctctttgttgggatttccccagggaccagcagttctcccccagcatgtgccggacttgtggcctgcagtcaccgtgtcctgagctgagagctgggacccgccgagggggtgcagagctcctacgtgaggatttctcaggagattaagccaaaactacagtacaaataccctgccaaatacaaactcttacaagatccgagggggaaatgcttctgcccactgccctgctccttgcctggccaaacctccaaggggtcagtgcctgctggagcagcaaccccagacgtgccaggaaaggaagggcctcagacagcccctccgtggcaccggggctctcttgggcagcagctcccggccacgcaggtccctgccagccccatgtccctgcccaaatcactgcccccatggtcacaaagccaagccccaggggcaaactgctgcaggctgccctgctgcagtggcacaagtgccaagatgggctgagggagggtggcttttcttttccttcccctctcgggaagctggaggctcagcgctcctgacagatctggcaaatggtgccctgaggctctggctggaaaataccgtgcaggggctcccagctgtgagctgggctctggcttcggaattcagactttggaggggggaactcactaattattgattaaaagattttgctaaaccatttccgtaagcatcagcccactgaggaaagcgtgctgaattatacatcacagcgccgtgctgccctgcctcccgcaacagggccgctttgcacccaaaacagggccttggcatggggtgaggcagtgccacccgtgggggcacggcagcgccctggctgacccccccacgttgacccccctgctgtggtgggacttgctaacgagcaaacgtgctgatggaaaacaaccccggggctccgcgcccagctgcagctctcgctctcgatctgcggtgtcctttggtgaaattttaaacagtcgccttttttttctttttcttttttttccccagcttgccattaatttcaaatcgagggggacctaatttagtaaatgacatgcttcaggaaggctttaattagctgcagacggaggcagctggtgctcccacggggtccggatcgcacagggggctgcgcgcccaggctgggggtcgtggtgctggctcagcgagggctgtgggtgggggggctgataaatgggggtagtttctctctaccaaaaggccaaatcccctccacatctgtgcccccaaccagccggggcgtgctctcatttcccacccatcaccccaaatctcactgccggtgagcctgggcatccccgccagctcgtgggagctcggcgggtcggggcgacgtggcagcagctcctgccatgcagcagcagcggggctggggcacagcagggcgggggcgtccccggccctgtcggcagctgtgagccacgcaaatagccgagtttcctggaggcaacagggctgaaactcgaaagcaagagccgggcgctcagaatagcctcagctggccggtgggagcaggcagaggggcagctcggcttggggtacagggtgagtgtgaggccctgggggctgcccggccctcccagcttttccaggaaggtgtggggcagcatctccagctgcggggggtctccagctgcggggacgcacagtgctgagagcccctggccccaccagcgccactgtgcagctgtgtggggtcggggcgtccccgagctaaatttactccccagtctccaatcaaaacttgcgctggggggcccgagctggcagaaaaaaggaactgaaacggtctccagcaagcgctgacgtgtgaagcagagcgagcgccgcgccaagcgagcgccgtgccgcgcgcctcgggctgggccgagctggcctgggctgggtcgagcttggcttctgcttggggatggcagcaccatgccagggtgggtgcccagcctgggcgacccagcagatgcacacacacacgtgcacacacaaacacgcacacacacacatgcacacacacacacacgcacaccctgcctcaccaagcgcacagggcccccaggatgtggcaccgccaaccccacacacccggggctgggcgcgctgctctgcccatggccctgtccctgtgtcccaccccctgcccaagctgggccccctgttcccactccaccactgggtcaccccgaatccttgctgacagacggaattactccagtaattcctgcctgcctccaggcagcgccatggctgagcttgtccacgacactgcgacctgccatgaaatgcatgaggcagactcaagaatcccaaaatacagagcagatttcccagaatgtgtgaaataaaatacattaggaatgttccccctgcagcagagccctggctgcaggcagctcagagagcgcagcttccattgcaccatgattaaaggtgaattaaatgaaattaataaatgcttcaggcgggctcagctgcaagtaccacctgaccgaaatcgcccgctcacagagaagagaaaatttgcatggcagtggcaaaacagcaaccgcccgctgtacagctgggcagcccgagcagccctgccactgggatgtttttttctggagccacgcactcagcccgcgcggaggtaaatagcaacaaatagaaactgcatggcttgtccaggtcataaacaagctgagccaactcaaaaataaacacctctgagatgcttaaaaatatttgtcatttcaagtttagcgtgttaataaagtaaaactcccaacagagctatgcacctgttctggaaaggaagcaaacacttgttttctgccggcgtttactgtaaaaagccgcgtttcctgatgcaggtgcgccaacaaagccatctcttgatgtcctgaaaacagcttgtctgaaaaatgaacgtgagttgagcttcatcgctgtttaagactcccatgagcgggtttgtagctttctgttgggacacatcgggagcaggcacggtgctggggttggggaggtggcggcggggaggcaggagcggggccgggcccagcgggtgtgcgatgtgtgcggcgatgtgctccgccagccaccacgtccctgccccaggcccccgggcatgagcaaggtgcttgtttcactgagccgtttggggaagtttttaagccaaatgctgatttttctccccactttgacagcctgacagggccagagctgtggcatgggggggaactcacgggaggtgatggggacactgtatgcatcccagggctggatcctgcaccccacaaagtgctgcccctgccagcacaccccatccccggacagacagacggcctttccccagccccctgcagcctccctgggccaagtgttgttttttaagtgtttctgccatgtttttcagcatttttagaagggcacctcgggctggccatcgcatgtacgtgcagctgcacttcaccagccccacagctgggcccgcgctcccccctggccagatgtgctgaccgcagccacggggctcaggggcagcactttatcttgctaaaaattgcatcctggccgaaaacaagtggccactttggccaaacaccagctggcggccaggaatagccgtggccacggcacgctcggcttcctcggaacaaactttccagcacgtggcaccgcgagcgccgctgcctggcatggcagccagcagggccaggagcaggagaagctcattttgggctccattttggcaatggctcagagagggggtttgcaggcagctccccagtgatggtgactggggtttgggaaaatggggggtgaagagtttttgccccccgactccagagcaggctctggtctcatttcgctgcagggcactgggggcatgtcagtgcagcagtttgtagaaacacctccaacactgcaaatgctgcaaagtgcagtggtaagacaaggagtaatggccttaaattaaaagagagtaggtttaggttagataccaggaaggaattgttcactcagtggcggtgaggctctgcaccaggctgcccagagaagctgtggctgccccttccctgaagagcccaaggccaggctggacgggcctttgggacacccggtgtggtgggaggcggtgtgcccatggcagggggctggggccagatgagcgttaaggtcccttcccacccaaaccatgaggtgattctaaaactgggccatgaaagtgcccatcctgcagccatgttgaacacaggctgaaccatgtatgcagtgaaattgtgtgtgtgtgtctgtgtgtgtgtgtgtgtgtctgtgtgtgtgtgtgtgtgtgtgtgtgtctgaggggggctcacatggccagagggctttgtgccgcactccccagagccacccggtgccactgtggccacagaggctggtcccctccagccacaggccagcagtgctcctgctgggcccagggccgctctgtgccacgctgctgcacagtgccccacagtttcctggggcctctgctgtgcccagccacacacaatgggcttcctgcactgctcctggccctgtccccctccccaggctcacccacgggcatgttcaaagccccttagtgtggggacaccacagggcacaggggacgctgccctagggcaaggtggcagggaccagcctgcagcccccacaccggccgtggctcgccaaggtcaggcacacagggacgagcgtggccccacacatgccggctggccccggccccagccgccctctcaccccctgcacaggggtctgtggctgccaggggtcccctgggctccctggatctgcagcgcacccacaccccagccctggctggctccgcgcaggcctgcagcaccgttttccccccagaaatgaagaaaagcccaggcagcccccctctgctgctagcagggcctgtgctcttactcccataacttcaaatgaggctggaagggcctggagcatcagcagggtaaggatgggtcaccgctctgtgtggctctgccccgaggcgcaggcagggcagagcaggcacagctgcagcagtttctgctggcctaaaattagcttctgggaaagggccacaaattccccgtgctgcaccccgtggggctgagatgtgccacgtccccatgacagggcccgggggaaggcaacgtggccacgtctgcacatacagcgaccacagggacacgtgctgggccaggattcggcccctggggctcacggtagagccgtgcaccatcacagagccagcctggccccatcctgctgcccccagctccctgcagccccccgtgccacgtacccatcaccatcaccactgaagggacacggagccggaggcagtgacggggccgggagctcgcctttattccagccccaacaggccccatagccccgcaacagggtggctgagccccacgtccacaccagccagggccgcagcaccctggcccagctgctgccccgaagtgcttttttttttttttttcctttttttccccatttttcctatgaaatcaagaatgttgcccttgcccttggcttctttgcaaaggttttgggggccaggaggcgacgggacgggggtgccggaccctatggggatggagagagggggctgagcagggccagcaggagcagcggggacaggaggtggccggggcagccgcggctggaggtggcgtccctggaggcgaagaccttgcggctgtggagcggctgcggcgggcggtagttgttggtcatcttcaggggcacggcgcccgagactgggaagtagacggtgttcacaaacgcccgcagctgccggggggagaggcgtggggttgggagccgtgccactgcgtgtcccaaccctggggcacgcgtggctcaaacacggggccaagtgaattcatccacccaccccgctcagctcccccagccccacagcccagcccagtgccccaggagcagacaccccctttacctcccccatgctattgttcaggtgtaaggaaacgcacaaggagcaggttgctcacggcacggcttttggccacctgaggctctccagccccacggcagggccgcccacgctacctgcgcccagccgatgtccacaggctcctcgaacacggtccagacgacggcctcgctgcagtcgggggtggtgagggagccctggtagcggtagtaccgggagagccggctgacgcgtggcagcagcgtgctcaggcggaaggtggaggccaggtccacagattgccctgcgtggtgagacgcgggcactcagcccacaccaggacaggggacagcagggccacccccatccgctgccccacaacccccccattgccaccagcccacccccaggcctctcccctcctgcttgtgccccggggcaggagcacgagggtgcctcaccagcatgggagatgttcctcaggccgctgacgatggtgttgtagttggggtttggggcttcagagacctgcaatgcaagagcaagcagctcaggtgggcagagcccatcccgcagcctggcagtggggtggaggagtttgcagcgaggcccctgctttatacgatgtcggggggctctcaggcagggatcgcccagagccggcatttcccaaggagcctttgggaacatatacgggtcctctggggacatttgccccctggtcgcagcagtgctctgcttgaacccacgggtgctggagggcaccaaccccatgagccaccaccagcctacctggaagaagcagcccaggacggccagcccgctcgggtgccccttggcctcccccagggtgcggtacttggcgttgatgtggacgatgtgcagctggggaaagacagaggggctgagcgtgggcacccagccgagcccctacctcaccctgacctcgccaggcactgggttgggagagcaacatgccaggggcagaggagggaaaggggatgcacagcccgtgtgcccgagcatctgctccagccgcagtgagtcagttttgctctactctgacagaccataaataccttacaggcacagaggctcgtttgtccaggagcatcccaaggtgtttggtggagccgcgctcacacccgccctccctcccttccccgacttgagacacgatcccttcgggcaccggggctgctccccgcctacctccatggggagctggtgcccgtccagggtgtgctcggagccgttcccggccgggctgccccagtggaagtggagctgcagcgcgcggtaccggccggggaggcccccgccgctgatggcgatgtgctcggaggccggctcaccctccaggctcagcatcactgcaagggacgggatagggacagggctggccccgcggggacaccggagagctgcagcaccagcagggggctgagcccatcgcgggggcagagcacccacccgtgtgcccgttgttcaccagcctccacttgcccgggggggcctggtcgtagccctcgaagacgatgtccccaaggccgctgtccctctgcagccggcgcctgtcgatgttcaccggggactgcttgctgccgccgcacgtggccttcagctcctttcagtggctggggcctgcgggggggcacggcc

SEQ ID NO: 119 - DNA, 219 bp, Cairina moschata
  misc_feature (1)..(219): 2.2-2.4 fragment homologue in Duck
actgtgcagctgtgtggggtcggggcgtccccgagctaaatttactccccagtctccaatcaaaacttgcgctggggggcccgagctggcagaaaaaaggaactgaaacggtctccagcaagcgctgacgtgtgaagcagagcgagcgccgcgccaagcgagcgccgtgccgcgcgcctcgggctgggccgagctggcctgggctgggtcgagcttggc